(19) United States
(12) Patent Application Publication
(10) Pub. No.: US 2025/0065080 A1
(43) Pub. Date: Feb. 27, 2025

(54) DEVICES AND METHODS FOR HOLLOW TISSUE ELECTROTRANSFER

(71) Applicant: Aldevron, LLC, South Fargo, ND (US)

(72) Inventors: Christopher MAGNIN, South Fargo, ND (US); Robert FARRA, South Fargo, ND (US); Jaka CEMAZAR, South Fargo, ND (US)

(21) Appl. No.: 18/721,403

(22) PCT Filed: Dec. 23, 2022
(86) PCT No.: PCT/US2022/082365

§ 371 Date: Jun. 18, 2024

Related U.S. Application Data
(60) Provisional application No. 63/293,365, filed on Dec. 23, 2021.

(51) Int. Cl. A61M 25/00; A61M 25/10; A61N 1/05; A61N 1/32

(52) U.S. Cl. CPC A61M 25/0082; A61M 25/0026; A61M 25/0068; A61M 25/0097; A61N 1/0519; A61N 1/327; A61M 2025/0002; A61M 2025/0046; A61M 2025/1013; A61M 2025/1061; A61M 2210/1032; A61M 2210/1035

(57) ABSTRACT

The invention provides devices and systems that help deliver agents of interest, such as therapeutic agents (e.g., nucleic acid vectors) to cells of hollow tissues, such as lung. Also provided herein are methods of using such devices, including methods of treating diseases by delivering therapeutic agents to hollow tissues.

### FIELD OF THE INVENTION

In general, the invention relates to methods of delivering agents to hollow tissues.

### BACKGROUND

Drug delivery to internal tissues, such as airway epithelia of the lung, represents a major hurdle for development of therapeutic strategies seeking to target such tissues. Delivery to the lung has proven challenging, despite apparent accessibility through the respiratory tract, because the lung has evolved a robust barrier function to protect the host from respiratory pathogens. Thus, there is a need in the field for improved methods of delivering therapeutic agents to hollow tissues, such as lung.

### SUMMARY OF THE INVENTION

The invention provides devices and systems that help deliver agents of interest, such as therapeutic agents (e.g., nucleic acid vectors) to cells of hollow tissues, such as lung. Also provided herein are methods of using such devices, including methods of treating diseases by delivering therapeutic agents to hollow tissues.

In general, the devices described herein include a catheter having a balloon and an electrode. The catheter may include a balloon positioned at or near the distal end of the catheter and a delivery lumen that runs from a proximal end of the catheter to a delivery port within the catheter. The electrode may be disposed on or about the catheter. The electrode may be connected to a lead disposed within or on the catheter. The device may further include an elongate conductor that runs through at least a portion of the catheter to the lead.

In one aspect, provided herein is a device comprising a catheter, a distal balloon, and an electrode. The catheter comprises: (i) a proximal end and a distal end; (ii) a delivery lumen that runs from the proximal end of the catheter to a delivery port proximal to the distal end of the catheter; (iv) an elongate conductor that runs from the proximal end of the catheter to a lead proximal to the distal end of the catheter; and (v) an inflation lumen that runs from the proximal end of the catheter to an inflation port proximal to the distal end of the catheter. The distal balloon is disposed about the catheter over the distal inflation port and distal to the delivery port, and distal airflow through the inflation lumen inflates the distal balloon. The electrode is disposed about the catheter proximal to the distal balloon, wherein the electrode is connected to the lead. In some embodiments, the catheter further comprises a bypass lumen that runs from the proximal end of the catheter (e.g., from the proximal end of the device) through the distal end of the catheter (e.g., through the distal end of the device).

In some embodiments, the electrode wraps around the circumference of the catheter. In some embodiments, application of a voltage to the electrode generates an electric field and a corresponding a current that flows radially relative to the catheter (e.g., the radial flow may be normal to the catheter surface or proximal or distal with a radial component). In some embodiments, the electrode is a mesh, a spiral, or a combination thereof. In some embodiments, the axial length of the electrode is from two-fold to 20-fold the outer diameter of the catheter (e.g., the axial length of the electrode is about five-fold the outer diameter of the catheter). In some embodiments, the electrode comprises multiple, electrically independent segments.

In some embodiments, the balloon is a stretching (e.g., compliant) balloon comprising an elastic material (e.g., polyurethane, thermoplastic elastomers, styrenic block copolymers, or isoprene rubber). In some embodiments, the axial length of the balloon is from one-fold to ten-fold the outer diameter of the catheter. In some embodiments, the balloon is radially inflatable to a diameter from 20% to 500% greater than the outer diameter of the balloon in its relaxed shape. In some embodiments, the balloon is a thin-walled tube (e.g., polyurethan tube, thermoplastic elastomers, styrenic block copolymers, or isoprene rubber) running from a proximal step down in the catheter to a distal step up in the catheter, wherein the outer diameter of the deflated balloon is substantially flush with the outer diameter of the catheter. In some embodiments, the balloon comprises a lubricious coating. In some embodiments, the balloon comprises a pressure sensor.

In some embodiments, the catheter comprises an inner tube and an outer tube, wherein one or more inner lumens are disposed within the inner tube and one or more outer lumens are disposed between the outer wall of the inner tube and the inner wall of the outer tube. In some embodiments, the elongate conductor is disposed within one of the one or more the outer lumens. In some embodiments, the one or more outer lumens comprises the inflation lumen. In some embodiments, the one or more inner lumens comprises the delivery lumen. In some embodiments, the one or more inner lumens comprises the bypass lumen.

In some embodiments, the elongate conductor is disposed within an insulating sleeve (e.g., a polyimide insulating sleeve). In some embodiments, the distal end of the catheter comprises an atraumatic tip. In some embodiments, the proximal end of the catheter is connected to one or more inlet ports.

In another aspect, the invention features a device comprising: (a) a catheter, wherein the catheter comprises: (i) a proximal end and a distal end; (ii) a delivery lumen that runs from the proximal end of the catheter to a delivery port proximal to the distal end of the catheter; (iv) an elongate conductor that runs from the proximal end of the catheter to a lead proximal to the distal end of the catheter; and (v) one or more inflation lumens that run from the proximal end of the catheter to a proximal inflation port proximal to the delivery port and a distal inflation port distal to the delivery port, wherein the distal inflation port is proximal to the distal end of the catheter; (b) a proximal balloon disposed about the catheter over the proximal inflation port; (c) a distal balloon disposed about the catheter over the distal inflation port, wherein distal airflow through the inflation lumen inflates the distal balloon; and (d) an electrode disposed about the catheter proximal to the balloon, wherein the electrode is connected to the lead. In some embodiments, the catheter further comprises: (iv) a bypass lumen that runs from the proximal end of the catheter through the distal end of the catheter. In some embodiments, the one or more inflation lumens comprises a proximal inflation lumen that runs to the proximal inflation port and a distal inflation lumen that runs to the distal inflation port. In some embodiments, the proximal balloon is independently inflatable relative to the distal balloon. In some embodiments, the catheter comprises a single inflation lumen that runs to both the proximal inflation port and the distal inflation port. In some embodiments, the electrode wraps around the circumference of the catheter. In some embodiments, application of a voltage to the electrode generates an electric field and a corresponding a current that flows radially relative to the catheter.

In some embodiments, the electrode is a mesh, a spiral, or a combination thereof. In some embodiments, the axial length of the electrode is from two-fold to 20-fold the outer diameter of the catheter (e.g., about five-fold the outer diameter of the catheter). In some embodiments, the electrode comprises multiple, electrically independent segments.

In some embodiments, the distal balloon and/or the proximal balloon are stretching (e.g., compliant) balloons comprising an elastic material, such as polyurethane, a thermoplastic elastomer, a styrenic block copolymer, or isoprene rubber. In some embodiments, the axial length of each of the distal balloon and/or the proximal balloon is from one-fold to ten-fold the outer diameter of the catheter. In some embodiments, each of the distal balloon and/or the proximal balloon is radially inflatable to a diameter from 20% to 500% greater than the outer diameter of distal balloon and/or proximal balloon in its relaxed shape. In some embodiments, the distal balloon and/or the proximal balloon are thin-walled polyurethane tubes running from a proximal step down in the catheter to a distal step up in the catheter, wherein the outer diameter of the deflated balloon is flush with the outer diameter of the catheter. In some embodiments, the distal balloon and/or the proximal balloon comprise a lubricious coating. In some embodiments, the distal balloon and/or the proximal balloon comprises a pressure sensor.

In some embodiments, the catheter comprises an inner tube and an outer tube, wherein one or more inner lumens are disposed within the inner tube and one or more outer lumens are disposed between the outer wall of the inner tube and the inner wall of the outer tube. In some embodiments, the elongate conductor is disposed within one of the one or more the outer lumens. In some embodiments, the one or more outer lumens comprises the distal inflation lumen and/or the proximal inflation lumen. In some embodiments, the one or more inner lumens comprises the delivery lumen. In some embodiments, the one or more inner lumens comprises the bypass lumen.

In some embodiments, the elongate conductor is disposed within an insulating sleeve (e.g., a polyimide insulating sleeve). In some embodiments, the distal end of the catheter comprises an atraumatic tip.

In some embodiments of any of the preceding aspects, the proximal end of the catheter is connected to one or more inlet ports.

In another aspect, the invention includes a system comprising the device of any one of the preceding aspects, wherein the elongate conductor is connected to a waveform generator.

In another aspect, the provided herein is a method of exposing a target area in a hollow tissue to electrical energy. The method includes: (a) providing a device having: (i) a catheter, wherein the catheter comprises: a proximal end and a distal end, a delivery lumen that runs from the proximal end of the catheter to a delivery port proximal to the distal end of the catheter, an elongate conductor that runs from the proximal end of the catheter to a lead proximal to the distal end of the catheter, and an inflation lumens that run from the proximal end of the catheter to an inflation port distal to the delivery port, wherein the distal inflation port is proximal to the distal end of the catheter; (ii) a balloon disposed about the catheter over the inflation port; and (iii) an electrode disposed about the catheter proximal to the balloon, wherein the electrode is connected to the lead; (b) positioning the electrode within the hollow tissue, wherein the region of the hollow tissue radially adjacent to the electrode comprises all or a portion of the target area; (c) flowing an inflation medium distally through the inflation lumen to inflate the balloon such that the balloon contacts the hollow tissue; (d) introducing an agent through the delivery lumen and the delivery port such that the agent contacts the target area; and (e) while the balloon is inflated, transmitting electrical energy through the electrode, thereby exposing the target area to the electrical energy. In some embodiments, the transmission of the electrical energy is at conditions suitable for electrotransfer of the agent into a target cell in the target area.

In another aspect of the invention, provided is a method of delivering an agent to a target cell in a hollow tissue, the method comprising: (a) providing a device comprising: (i) a catheter, wherein the catheter comprises: a proximal end and a distal end, a delivery lumen that runs from the proximal end of the catheter to a delivery port proximal to the distal end of the catheter, an elongate conductor that runs from the proximal end of the catheter to a lead proximal to the distal end of the catheter, and an inflation lumens that run from the proximal end of the catheter to an inflation port distal to the delivery port, wherein the distal inflation port is proximal to the distal end of the catheter; (ii) a balloon disposed about the catheter over the inflation port; and (iii) an electrode disposed about the catheter proximal to the balloon, wherein the electrode is connected to the lead; (b) positioning the electrode within the hollow tissue, wherein the region of the hollow tissue radially adjacent to the electrode comprises the target cell; (c) flowing an inflation medium distally through the inflation lumen to inflate the balloon such that the balloon contacts the hollow tissue; (d) introducing the agent through the delivery lumen and the delivery port such that the agent contacts the region of the hollow tissue radially adjacent to the electrode; and (e) while the balloon is inflated, transmitting electrical energy through the electrode at conditions suitable for electrotransfer of the agent into the target cell. In some embodiments, the agent is a therapeutic agent. In some embodiments, the agent is a nucleic acid vector. In some embodiments, the nucleic acid vector is expressed in the hollow tissue.

In another aspect, the invention provides a method of expressing a nucleic acid vector in a target cell in a hollow tissue. The method includes: (a) providing a device comprising: (i) a catheter, wherein the catheter comprises: a proximal end and a distal end, a delivery lumen that runs from the proximal end of the catheter to a delivery port proximal to the distal end of the catheter, an elongate conductor that runs from the proximal end of the catheter to a lead proximal to the distal end of the catheter, and an inflation lumens that run from the proximal end of the catheter to an inflation port distal to the delivery port, wherein the distal inflation port is proximal to the distal end of the catheter; (ii) a balloon disposed about the catheter over the inflation port; and (iii) an electrode disposed about the catheter proximal to the balloon, wherein the electrode is connected to the lead; (b) positioning the electrode within the hollow tissue, wherein the region of the hollow tissue radially adjacent to the electrode comprises the target cell; (c) flowing an inflation medium distally through the inflation lumen to inflate the balloon such that the balloon contacts the hollow tissue; (d) introducing the nucleic acid vector through the delivery lumen and the delivery port such that the nucleic acid vector contacts the region of the hollow tissue radially adjacent to the electrode; (e) while the balloon is inflated, transmitting electrical energy through the electrode at conditions suitable for electrotransfer of the nucleic acid vector into the target cell; and (f) detecting expression of the nucleic acid vector by the target cell.

In another aspect, the invention includes a method of treating a disease or disorder in an individual. The method includes: (a) providing a device comprising: (i) a catheter, wherein the catheter comprises: a proximal end and a distal end, a delivery lumen that runs from the proximal end of the catheter to a delivery port proximal to the distal end of the catheter, an elongate conductor that runs from the proximal end of the catheter to a lead proximal to the distal end of the catheter, and an inflation lumens that run from the proximal end of the catheter to an inflation port distal to the delivery port, wherein the distal inflation port is proximal to the distal end of the catheter; (ii) a balloon disposed about the catheter over the inflation port; and (iii) an electrode disposed about the catheter proximal to the balloon, wherein the electrode is connected to the lead; (b) positioning the electrode within a hollow tissue in the individual, wherein the region of the hollow tissue radially adjacent to the electrode comprises a target cell; (c) flowing an inflation medium distally through the inflation lumen to inflate the balloon such that the balloon contacts the hollow tissue; (d) introducing an effective amount of therapeutic agent through the delivery lumen and the delivery port such that the therapeutic agent contacts the region of the hollow tissue radially adjacent to the electrode; and (e) while the balloon is inflated, transmitting electrical energy through the electrode at conditions suitable for electrotransfer of an effective amount of the therapeutic agent into the target cell, thereby treating the disease or disorder. In some embodiments, the therapeutic agent is a nucleic acid vector. In some embodiments, the nucleic acid vector comprises a therapeutic protein-encoding sequence. In some embodiments, the therapeutic protein is a therapeutic replacement protein. In some embodiments, the individual is monitored for progression of the disease or disorder after the treatment.

In another aspect, the invention involves a method of exposing a target area in a hollow tissue to electrical energy. The method includes: (a) providing a device comprising: (i) a catheter, wherein the catheter comprises: a proximal end and a distal end; a delivery lumen that runs from the proximal end of the catheter to a delivery port proximal to the distal end of the catheter; an elongate conductor that runs from the proximal end of the catheter to a lead proximal to the distal end of the catheter; and one or more inflation lumens that run from the proximal end of the catheter to a proximal inflation port proximal to the delivery port and a distal inflation port distal to the delivery port, wherein the distal inflation port is proximal to the distal end of the catheter; (ii) a proximal balloon disposed about the catheter over the proximal inflation port; (iii) a distal balloon disposed about the catheter over the distal inflation port; and (iv) an electrode disposed about the catheter proximal to the balloon, wherein the electrode is connected to the lead; (b) positioning the electrode within the hollow tissue, wherein the region of the hollow tissue radially adjacent to the electrode comprises all or a portion of the target area; (c) flowing an inflation medium distally through the one or more inflation lumens to inflate the proximal balloon and the distal balloon such that each of the proximal balloon and distal balloon contacts the hollow tissue; (d) introducing an agent through the delivery lumen and the delivery port such that the agent contacts the target area; and (e) while the proximal and distal balloons are inflated, transmitting electrical energy through the electrode, thereby exposing the target area to the electrical energy. In some embodiments, the transmission of the electrical energy is at conditions suitable for electrotransfer of the agent into a target cell in the target area.

In another aspect of the invention, provided is a method of delivering an agent to a target cell in a hollow tissue, the method comprising: (a) providing a device comprising: (i) a catheter, wherein the catheter comprises: a proximal end and a distal end; a delivery lumen that runs from the proximal end of the catheter to a delivery port proximal to the distal end of the catheter; an elongate conductor that runs from the proximal end of the catheter to a lead proximal to the distal end of the catheter; and one or more inflation lumens that run from the proximal end of the catheter to a proximal inflation port proximal to the delivery port and a distal inflation port distal to the delivery port, wherein the distal inflation port is proximal to the distal end of the catheter; (ii) a proximal balloon disposed about the catheter over the proximal inflation port; (iii) a distal balloon disposed about the catheter over the distal inflation port; and (iv) an electrode disposed about the catheter proximal to the balloon, wherein the electrode is connected to the lead; (b) positioning the electrode within the hollow tissue, wherein the region of the hollow tissue radially adjacent to the electrode comprises the target cell; (c) flowing an inflation medium distally through the one or more inflation lumens to inflate the proximal balloon and the distal balloon such that each of the proximal balloon and distal balloon contacts the hollow tissue; (d) introducing the agent through the delivery lumen and the delivery port such that the agent contacts the region of the hollow tissue radially adjacent to the electrode; and (e) while the proximal and distal balloons are inflated, transmitting electrical energy through the electrode at conditions suitable for electrotransfer of the agent into the target cell. In some embodiments, the agent is a therapeutic agent. In some embodiments, the agent is a nucleic acid vector. In some embodiments, the nucleic acid vector is expressed in the hollow tissue.

In another aspect, the invention features a method of expressing a nucleic acid vector in a target cell in a hollow tissue. The method includes: (a) providing a device comprising: (i) a catheter, wherein the catheter comprises: a proximal end and a distal end; a delivery lumen that runs from the proximal end of the catheter to a delivery port proximal to the distal end of the catheter; an elongate conductor that runs from the proximal end of the catheter to a lead proximal to the distal end of the catheter; and one or more inflation lumens that run from the proximal end of the catheter to a proximal inflation port proximal to the delivery port and a distal inflation port distal to the delivery port, wherein the distal inflation port is proximal to the distal end of the catheter; (ii) a proximal balloon disposed about the catheter over the proximal inflation port; (iii) a distal balloon disposed about the catheter over the distal inflation port; and (iv) an electrode disposed about the catheter proximal to the balloon, wherein the electrode is connected to the lead; (b) positioning the electrode within the hollow tissue, wherein the region of the hollow tissue radially adjacent to the electrode comprises the target cell; (c) flowing an inflation medium distally through the one or more inflation lumens to inflate the proximal balloon and the distal balloon such that each of the proximal balloon and distal balloon contacts the hollow tissue; (d) introducing the nucleic acid vector through the delivery lumen and the delivery port such that the nucleic acid vector contacts the region of the hollow tissue radially adjacent to the electrode; (e) while the proximal and distal balloons are inflated, transmitting electrical energy through the electrode at conditions suitable for electrotransfer of the nucleic acid vector into the target cell; and (f) detecting expression of the nucleic acid vector by the target cell.

In another aspect, provided is a method of treating a disease or disorder in an individual. The method includes: (a) providing a device comprising: (i) a catheter, wherein the catheter comprises: a proximal end and a distal end; a delivery lumen that runs from the proximal end of the catheter to a delivery port proximal to the distal end of the catheter; an elongate conductor that runs from the proximal end of the catheter to a lead proximal to the distal end of the catheter; and one or more inflation lumens that run from the proximal end of the catheter to a proximal inflation port proximal to the delivery port and a distal inflation port distal to the delivery port, wherein the distal inflation port is proximal to the distal end of the catheter; (ii) a proximal balloon disposed about the catheter over the proximal inflation port; (iii) a distal balloon disposed about the catheter over the distal inflation port; and (iv) an electrode disposed about the catheter proximal to the balloon, wherein the electrode is connected to the lead; (b) positioning the electrode within a hollow tissue in the individual, wherein the region of the hollow tissue radially adjacent to the electrode comprises a target cell; (c) flowing an inflation medium distally through the one or more inflation lumens to inflate the proximal balloon and the distal balloon such that each of the proximal balloon and distal balloon contacts the hollow tissue; (d) introducing an effective amount of a therapeutic agent through the delivery lumen and the delivery port such that the therapeutic agent contacts the region of the hollow tissue radially adjacent to the electrode; and (e) while the proximal and distal balloons are inflated, transmitting electrical energy through the electrode at conditions suitable for electrotransfer of an effective amount of the therapeutic agent into the target cell, thereby treating the disease or disorder. In some embodiments, the therapeutic agent is a nucleic acid vector. In some embodiments, the nucleic acid vector comprises a therapeutic protein-encoding sequence. In some embodiments, the therapeutic protein is a therapeutic replacement protein. In some embodiments, the individual is monitored for progression of the disease or disorder after the treatment. In some embodiments, the one or more inflation lumens comprises a proximal inflation lumen that runs to the proximal inflation port and a distal inflation lumen that runs to the distal inflation port.

In some embodiments, the proximal balloon and the distal balloon are inflated simultaneously. In some embodiments, the proximal balloon is inflated independently from the distal balloon. In some embodiments, the proximal balloon is inflated after the distal balloon. In some embodiments, the catheter comprises a single inflation lumen that runs to both the proximal inflation port and the distal inflation port.

In some embodiments, the hollow tissue is a tubular tissue. In some embodiments, the catheter further comprises a bypass lumen that runs from the proximal end of the catheter through the distal end of the catheter, wherein the method is performed without occluding flow through the tubular tissue. In some embodiments, the hollow tissue is an airway, such as a trachea or a bronchiole. In some embodiments, the target cell is an airway epithelial cell. In some embodiments, the agent is formulated as a liquid, e.g., a viscous liquid.

### BRIEF DESCRIPTION OF THE DRA WINGS

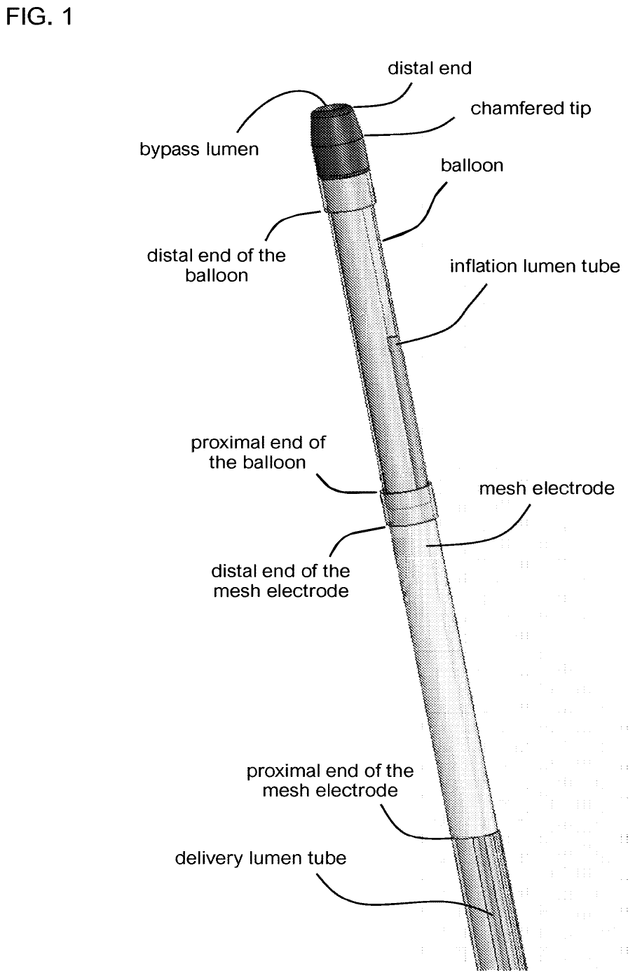

FIG. 1 is a slightly oblique side view drawing of a device of the invention having a single balloon and an electrode disposed about a catheter. The device features a central bypass tube and two peripheral tubes having an inflation lumen and a delivery lumen.

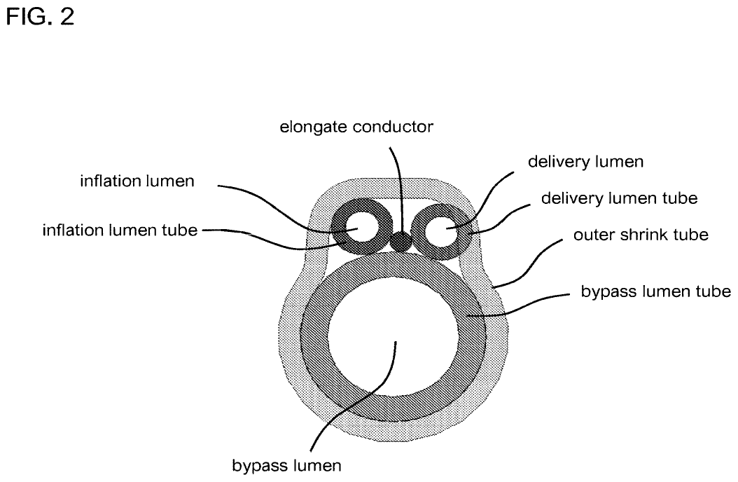

FIG. 2 is a transverse cross section drawing of the device of FIG. 1.

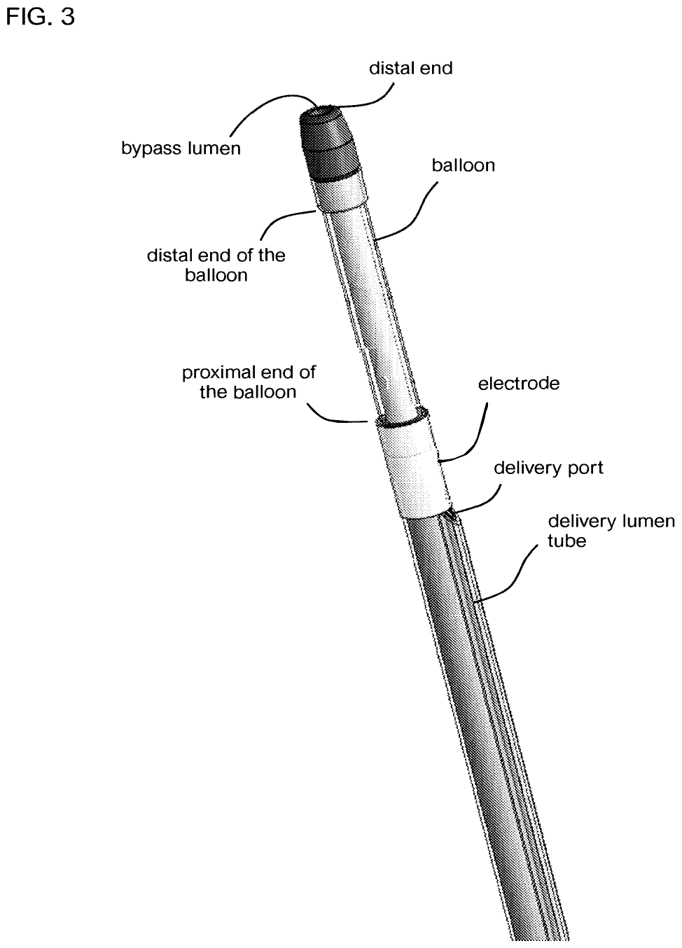

FIG. 3 is a slightly oblique side view drawing of a device of the invention having a single balloon and an electrode disposed about a catheter. The device features two central tubes (an inner tube to provide a bypass lumen and an outer tube to provide an inflation lumen) and a single peripheral tube to provide a delivery lumen.

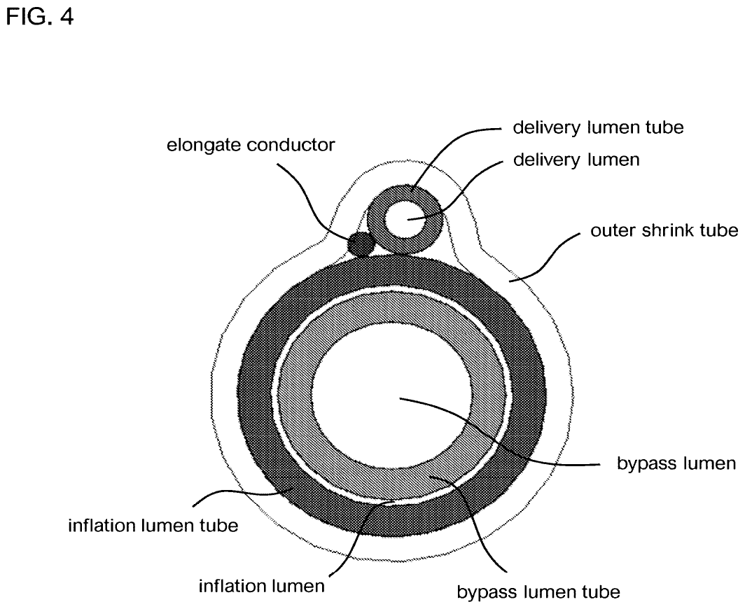

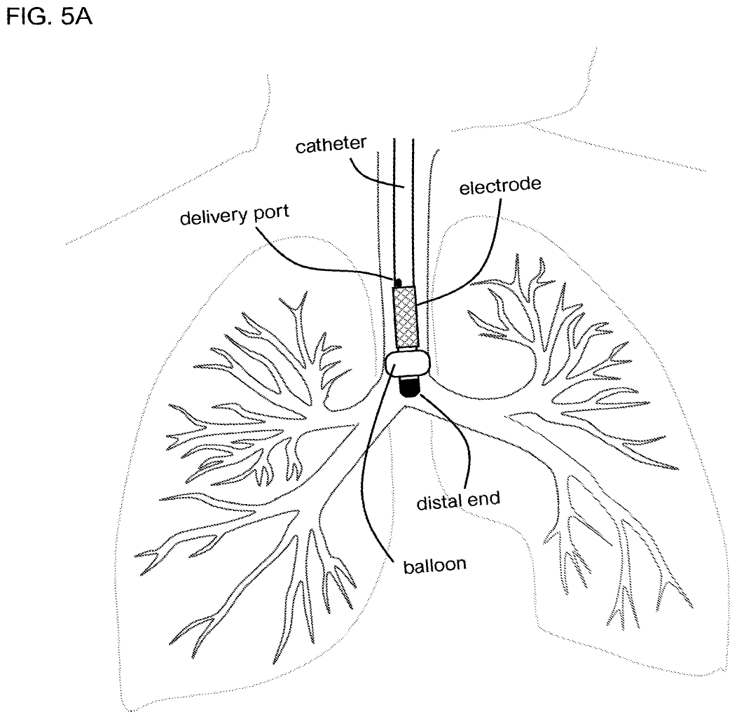

FIG. 4 is a transverse cross section drawing of the device of FIG. 3.

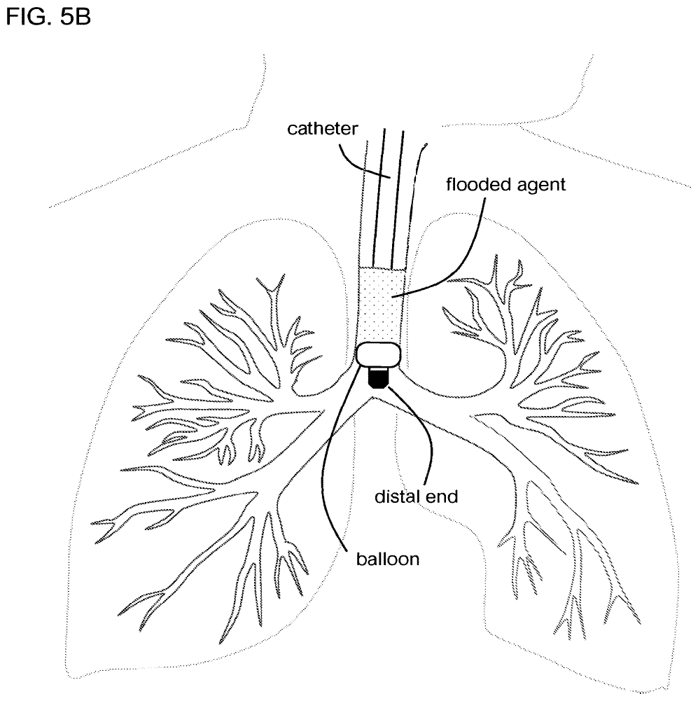

FIGS. 5A and 5B are schematic drawings of a single-balloon device positioned for electrotransfer to the trachea. FIG. 5A shows the device after inflation of the balloon, before introducing the agent. FIG. 5B shows the device after inflation of the balloon and after introducing the agent, which floods the trachea at the target area.

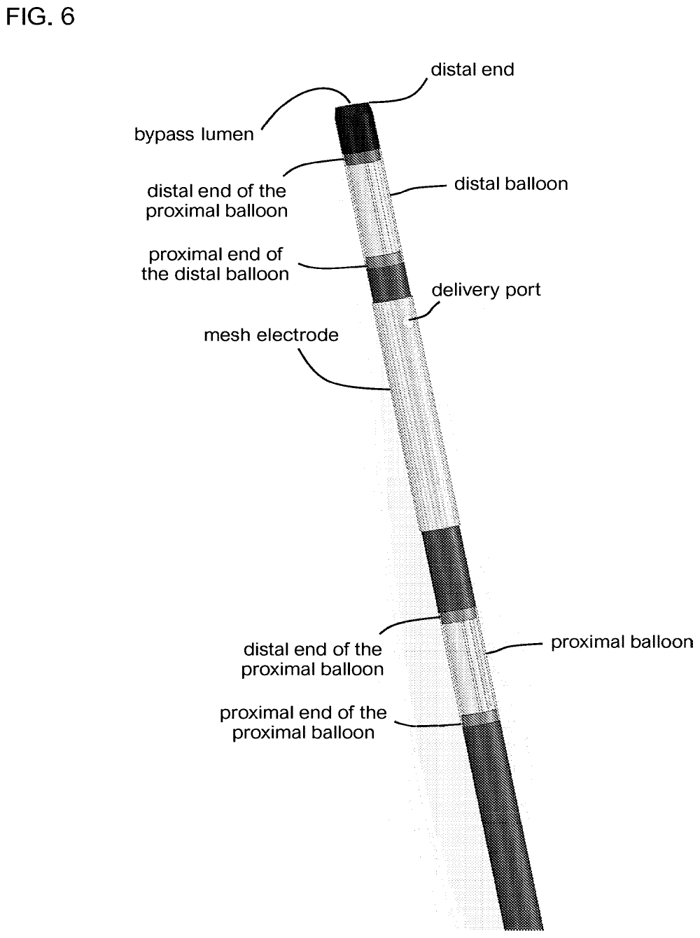

FIG. 6 is a side view drawing of a dual-balloon device of the invention having a proximal balloon disposed about a catheter, a distal balloon disposed about the catheter, and an electrode disposed about the catheter between the proximal and distal electrodes. The device features a single inflation lumen providing access to both balloons.

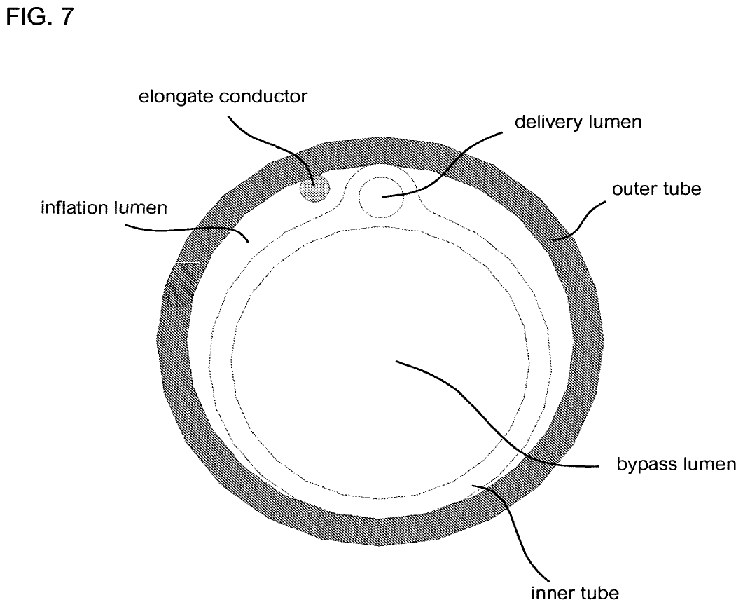

FIG. 7 is a transverse cross section drawing of the device of FIG. 6, showing the semi-annular inflation lumen between the outer tube and the inner tube.

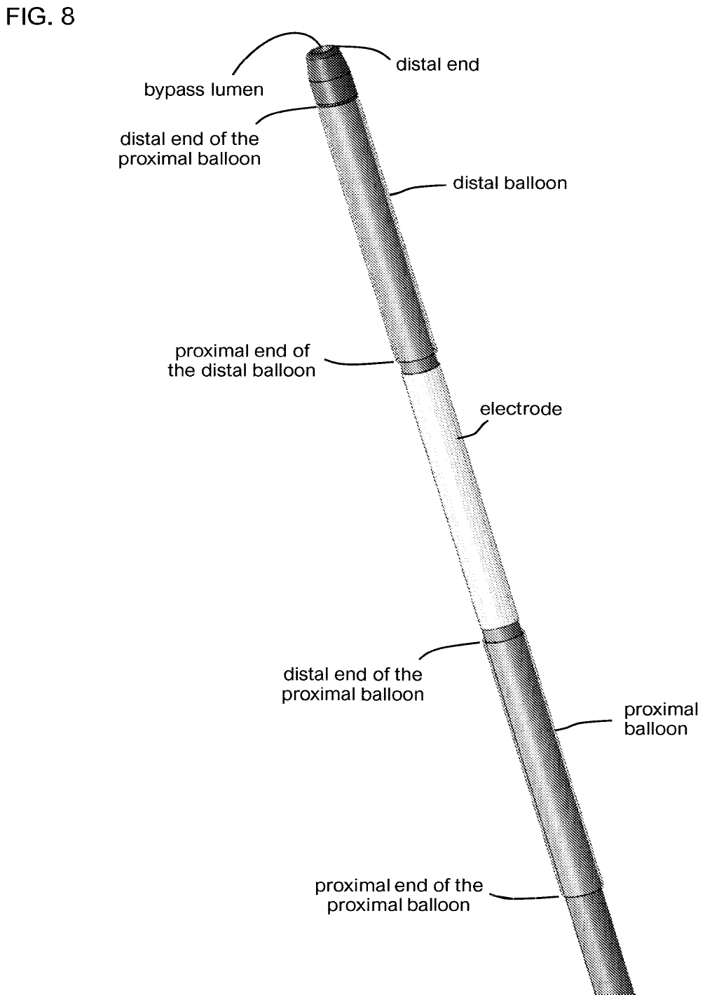

FIG. 8 is a slightly oblique side view drawing of a dual-balloon device of the invention having a proximal balloon disposed about a catheter, a distal balloon disposed about the catheter, and an electrode disposed about the catheter between the proximal and distal electrodes. The device features two separate inflation lumens providing access to the proximal and distal balloons.

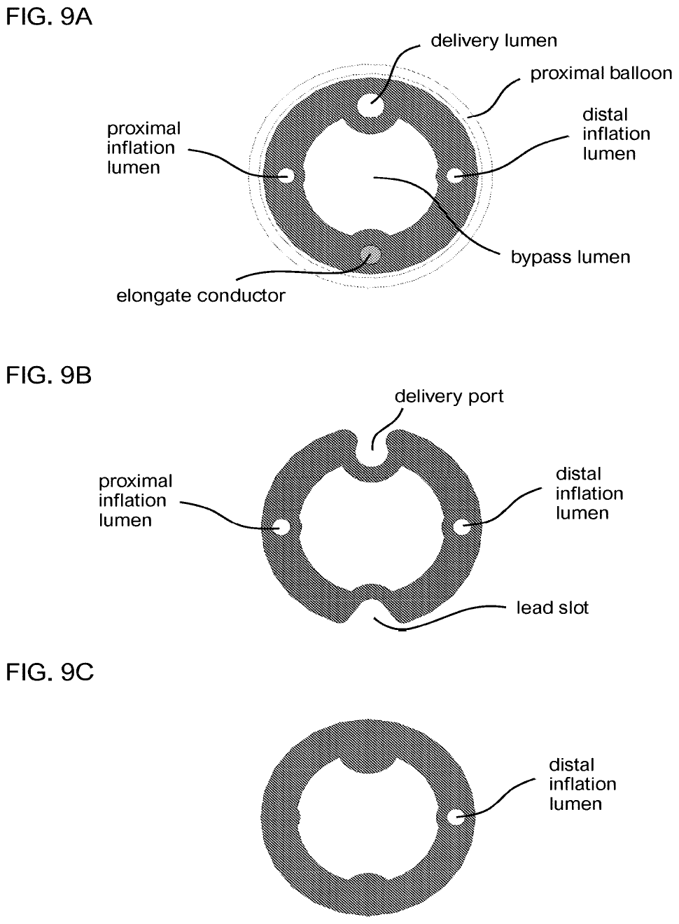

FIGS. 9A-9C are transverse cross section drawings of the device of FIG. 8. FIG. 9A is a transverse cross section at the proximal balloon. FIG. 9B is a transverse cross section at the electrode. FIG. 9C is a transverse cross section at the distal balloon.

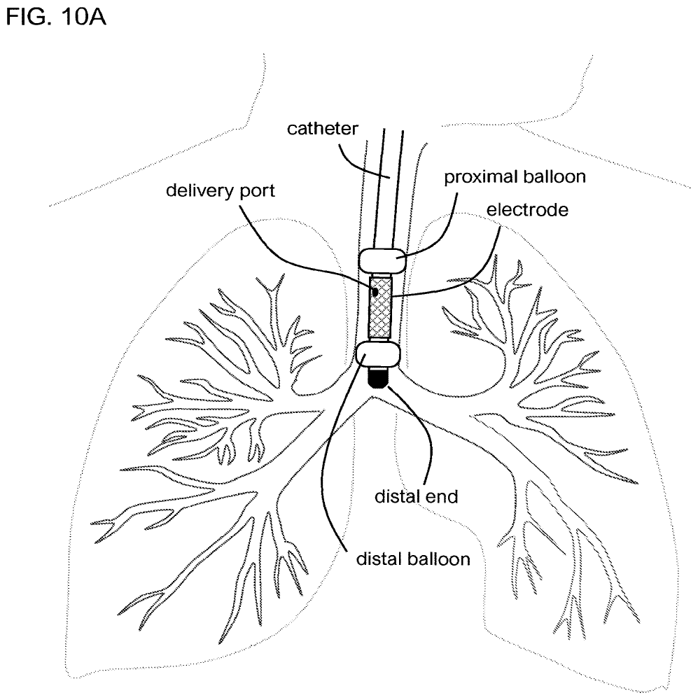

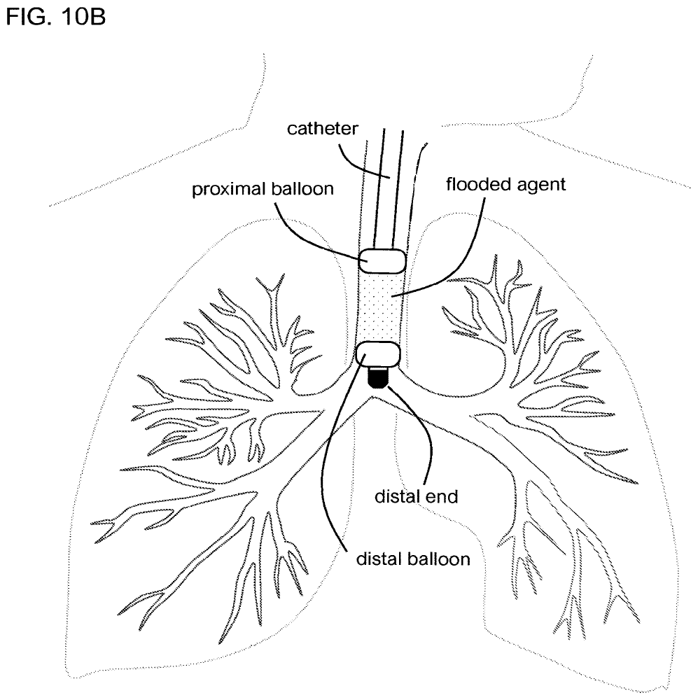

FIGS. 10A and 10B are schematic drawings of a dual-balloon device positioned for electrotransfer to the trachea. FIG. 10A shows the device after inflation of the balloons, before introducing the agent. FIG. 10B shows the device after inflation of the balloons and after introducing the agent, which floods the trachea at the target area between the proximal and distal balloons.

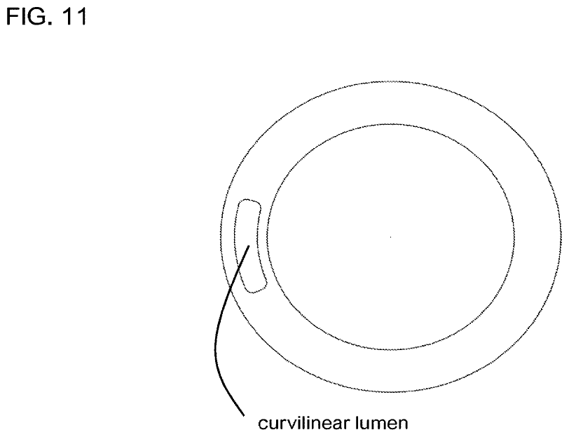

FIG. 11 is a transverse cross-section showing a curvilinear shape of the delivery lumen.

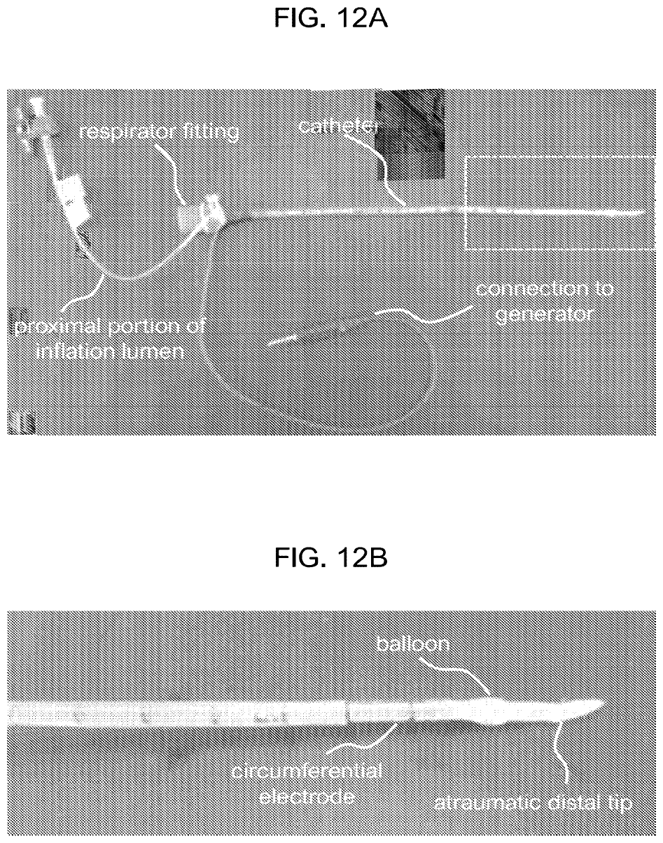

FIGS. 12A and 12B are photographs of a single-balloon device. FIG. 12A shows the full device including the proximal end of the catheter, which includes connections for power supply, inflation, and agent delivery. FIG. 12B is a blow-up view of the white dashed box of FIG. 12A, showing the circumferential electrode positioned immediately proximal to the balloon. Also shown is the atraumatic distal tip.

### DETAILED DESCRIPTION

Provided herein are devices and methods for delivering agents of interest, such as therapeutic agents (e.g., nucleic acid vectors) to hollow tissues, such as lung. Such devices and methods can be useful in treating diseases associated with dysfunction of hollow tissues, such as respiratory diseases.

### I. Definitions

Unless defined otherwise, technical, and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, a “hollow tissue” refers to a tissue that surrounds, in at least one dimension, a gas and/or liquid. The hollow tissue may include one or more layers (e.g., epithelial layers, including basement membranes) that line body cavities or lumens. In some instances, a hollow tissue is a tubular or luminal tissue, such as an airway (e.g., trachea, larynx, bronchus (e.g., main bronchus), or bronchiole), blood vessel (e.g., artery or vein), or gastrointestinal tract tissue (e.g., esophagus, stomach, duodenum, jejunum, colon, or rectum). As used herein, a target area of a hollow tissue may include all or a portion of the perimeter of the tissue (e.g., all or a portion of the epithelium).

As used herein, a “proximal end” of a catheter refers to the end of the catheter configured to be positioned closer to an operator relative to the distal end when the electrode is at or near the target area. In some instances, the proximal end is the proximal entry point of a material (e.g., an agent, an inflation medium, etc.) into a lumen of a catheter. In some instances, the proximal end of the catheter is different from the proximal end of the device. For instance, another element of the device (e.g., an inlet port) may extend further in the proximal direction than the catheter.

As used herein, a “distal end” of a catheter refers to the end of the catheter configured to be positioned further from an operator relative to the proximal end when the electrode is at or near the target area. In certain instances, the distal end of the catheter is the same point as the distal end of the device (i.e., the distal end of the catheter is the distal-most point of the device). In other instances, the distal end of the catheter is different from the distal end of the device, for example, where another element of the device (e.g., a separate atraumatic tip) runs further in the distal direction than the catheter.

As used herein, the term “axial” refers to the proximal-to-distal dimension taken along the length of a catheter. Thus, “axial length” refers to the proximal-to-distal distance from one point to another.

As used herein, the term “radial” refers to a direction perpendicular to the axial dimension. A radial vector has a radial component. For instance, current that flows radially relative to a cylindrical electrode and/or a catheter may flow in a direction perpendicular (e.g., normal) to the cylindrical electrode and/or a catheter or, alternatively, may have a radial component in addition to an axial component (e.g., the radial flow is both away from the catheter and proximal or distal to the electrode).

The term “radially adjacent” refers to a point that is offset from a reference point along the radial dimension and not the axial dimension. Thus, a region of hollow tissue that is radially adjacent to an electrode therewithin includes a region of tissue having the same axial length as the electrode and does not include any region of tissue proximal or distal to the electrode.

As used herein, “electrotransfer” refers to movement of a molecule (e.g., a nucleic acid, e.g., a naked nucleic acid) across a membrane of a target cell (e.g., from outside to inside the target cell) that is caused by transmission of an electric field (e.g., a pulsed electric field) to the microenvironment in which the cell resides. Electrotransfer may occur as a result of electrophoresis, i.e., movement of a molecule (e.g., a nucleic acid, e.g., a naked nucleic acid) along an electric field (e.g., in the direction of current), based on a charge of the molecule. Electrophoresis can induce electrotransfer, for example, by moving a molecule (e.g., a nucleic acid, e.g., a naked nucleic acid) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, i.e., generation of pores in the target cell caused by transmission of an electric field (e.g., a pulsed electric field), wherein the size, shape, and duration of the pores are suitable to accommodate movement of a molecule (e.g., a nucleic acid, e.g., a naked nucleic acid) from outside the target cell to inside the target cell. Thus, in some instances, electrotransfer occurs as a result of a combination of electrophoresis and electroporation.

As used herein, the term “circular DNA vector” refers to a DNA molecule in a circular form. Such circular form is typically capable of being amplified into concatemers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeably herein with the terms “covalently closed and circular DNA vector” and “c3DNA.” A skilled artisan will understand that such circular vectors include vectors that are covalently closed with supercoiling and complex DNA topology, as is described herein. In particular embodiments, a circular DNA vector is supercoiled (e.g., monomeric supercoiled). In certain instances, a circular DNA vector lacks a bacterial origin of replication.

As used herein, the term “therapeutic sequence” refers to the portion of a DNA molecule that contains any genetic material required for transcription in a target cell of one or more therapeutic moieties, which may include one or more coding sequences, promoters, terminators, introns, and/or other regulatory elements. A therapeutic moiety can be a therapeutic protein (e.g., a replacement protein (e.g., a protein that replaces a defective protein in the target cell) or an endogenous protein) and/or a therapeutic nucleic acid (e.g., one or more microRNAs). In DNA vectors having more than one transcription unit, the therapeutic sequence contains the plurality of transcription units. A therapeutic sequence may include one or more genes (e.g., heterologous genes or transgenes) to be administered for a therapeutic purpose.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides (e.g., polypeptides), native proteins, recombinant proteins, and fragments thereof. In some embodiments, a protein has a primary structure and no secondary, tertiary, or quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

As used herein, the term “therapeutic protein” refers to a protein that can treat a disease or disorder in a subject. In some embodiments, a therapeutic protein is a therapeutic replacement protein administered to replace a defective (e.g., mutated) protein in a subject. In some embodiments, a therapeutic protein is the same or functionally similar to a native protein that is not defective in a subject (e.g., cystic fibrosis transmembrane regulator (CFTR)).

As used herein, the term “therapeutic replacement protein” refers to a protein that is structurally similar to (e.g., structurally identical to) a protein that is endogenously expressed by a normal (e.g., healthy) individual. A therapeutic replacement protein can be administered to an individual that suffers from a disorder associated with a dysfunction of (or lack of) the protein to be replaced. In some embodiments, the therapeutic replacement protein corrects a defect in a protein resulting from a mutation (e.g., a point mutation, an insertion mutation, a deletion mutation, or a splice variant mutation) in the gene encoding the protein. Therapeutic replacement proteins do not include non-endogenous proteins, such as proteins associated with a pathogen (e.g., as part of a vaccine). Therapeutic replacement proteins may include enzymes, growth factors, hormones, interleukins, interferons, cytokines, anti-apoptosis factors, anti-diabetic factors, coagulation factors, anti-tumor factors, liver-secreted proteins, or neuroprotective factors.

As used herein, the term “therapeutic nucleic acid” refers to a nucleic acid that binds to (e.g., hybridizes with) a molecule (e.g., protein or nucleic acid) in the subject to confer its therapeutic effect (i.e., without necessarily being transcribed or translated). Therapeutic nucleic acids can be DNA or RNA, such as small interfering RNA (siRNA), short hairpin RNA (shRNA), microRNA (miRNA), a CRISPR molecule (e.g., guide RNA (gRNA)), an oligonucleotide (e.g., an antisense oligonucleotide), an aptamer, or a DNA vaccine. In some embodiments, the therapeutic nucleic acid may be a non-inflammatory or a non-immunogenic therapeutic nucleic acid. In other embodiments, the therapeutic nucleic acid is recognizable by the immune system (e.g., adaptive immune system) and may induce an immune response (e.g., an innate immune response). Such therapeutic nucleic acids include toll-like receptor (TLR) agonists.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector.

As used herein, a “vector” refers to a nucleic acid molecule capable of carrying a sequence of interest (e.g., a therapeutic sequence) to which is it linked into a target cell in which the sequence of interest can then be transcribed, replicated, processed, and/or expressed in the target cell. After a target cell or host cell processes the sequence of interest (e.g., a therapeutic sequence) of the vector, the sequence of interest (e.g., a therapeutic sequence) is not considered a vector. One type of vector is a “plasmid”, which refers to a circular double stranded DNA loop containing a bacterial backbone into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”).

As used herein, the term “individual” includes any mammal in need of treatment or prophylaxis, e.g., by a synthetic circular DNA vector described herein. In some embodiments, the individual is a human. In other embodiments, the individual is a non-human mammal (e.g., a non-human primate (e.g., a monkey), a mouse, a pig, a rabbit, a cat, or a dog). In some embodiments, the individual is human (e.g., a human patient). The individual may be male or female.

As used herein, “delivering,” “to deliver,” and grammatical variations thereof, is meant causing an agent (e.g., a therapeutic agent) to access a target cell. The agent can be delivered by administration of the agent to an individual having the target cell (e.g., locally administering the agent) such that the agent gains access to the organ or tissue in which the target cell resides. Additionally, or alternatively, the agent can be delivered by applying a stimulus to a tissue or organ harboring the agent, wherein the stimulus causes the agent to enter the target cell. Thus, in some instances, an agent is delivered to a target cell by transmitting an electric field into a tissue harboring the agent at conditions suitable for electrotransfer of the agent into a target cell within the tissue.

As used herein, “administering” is meant a method of giving a dosage of an agent or a composition thereof to an individual. The compositions utilized in the methods described herein can be administered through a delivery lumen and delivery port positioned in a hollow tissue (e.g., near a target area).

As used herein, an “effective amount” or “effective dose” of an agent refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, therapeutic circular DNA vectors of the invention are used to delay development of a disease or to slow the progression of a disease.

The terms “level of expression” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a sequence of interest (e.g., a therapeutic sequence), or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”). A sequence of interest (e.g., a therapeutic sequence), or functional portion thereof, may be expressible if it is not silenced, e.g., by DNA methylation and/or histone methylation and compaction. Expression persistence can be assessed by detecting or quantifying (i) mRNA transcribed from the sequence of interest (e.g., a therapeutic sequence) in the target cell or progeny thereof (e.g., through qPCR, RNA-seq, or any other suitable method) and (ii) protein translated from the sequence of interest (e.g., a therapeutic sequence) in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) in conjunction with either or both of (i) mRNA transcribed from the sequence of interest (e.g., a therapeutic sequence) in the target cell or progeny thereof and (ii) protein translated from the sequence of interest (e.g., a therapeutic sequence) in the target cell or progeny thereof. Expression persistence of a sequence of interest (e.g., a therapeutic sequence), or a functional portion thereof, can be quantified relative to a reference vector, such as a control vector produced in bacteria (e.g., a circular vector produced in bacteria or having one or more bacterial signatures not present in the vector of the invention (e.g., a plasmid)), using any gene expression characterization method known in the art. Expression persistence can be quantified at any given time point following administration of the vector. For example, in some embodiments, expression of a therapeutic circular DNA vector of the invention persists for at least two weeks after administration if it is detectable in the target cell, or progeny thereof, two weeks after administration of the therapeutic circular DNA vector. In some embodiments, expression of a gene “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a sequence of interest (e.g., a therapeutic sequence) is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

As used herein, the term “cystic fibrosis transmembrane regulator (CFTR)” refers to any native CFTR from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of known CFTR signaling (e.g., ion and water secretion and absorption in epithelial tissues). CFTR encompasses full-length, unprocessed CFTR, as well as any form of CFTR that results from native processing in the cell. An exemplary human CFTR sequence is provided as National Center for Biotechnology Information (NCBI) Gene ID: 1080.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within ±10% variability from the reference value, unless otherwise specified.

### II. Devices

Devices of the invention include catheters configured for insertion into a hollow tissue, e.g., a tubular tissue, such as an airway, gastrointestinal tract, or blood vessel. Catheters described herein can be a flexible or semi-rigid elongate shaft with one or more lumens (e.g., one or more delivery lumens and/or one or more inflation lumens) and one or more electrodes (e.g., an electrode disposed about the shaft), which allows administration of an agent to the hollow tissue and transmission of electrical energy to the hollow tissue exposed to the agent. Such devices can be used to perform electrotransfer of the agent into a target cell in the hollow tissue. In some embodiments, the catheter includes one or more (e.g., one, two, or more) balloons disposed about its distal region, which can be configured to control the area of hollow tissue exposed to the agent (e.g., to colocalize the agent with the target area to be exposed to the electrical energy transmitted by the electrode). In general, catheters of the invention may include inlet ports for agents, balloon inflation, conductors, etc. In some embodiments, such devices are connected to a waveform generator and/or power supply as part of a system (e.g., a system for electrotransfer to hollow tissues).

In general, the devices described herein include a catheter having one or more balloons and an electrode. The catheter may include a balloon positioned at or near the distal end of the catheter and a delivery lumen that runs from a proximal end of the catheter to a delivery port within the catheter. The electrode may be disposed on or about the catheter. The electrode may be connected to a lead disposed within or on the catheter. The device may further include an elongate conductor that runs through at least a portion of the catheter to the lead.

### Catheters

Electrotransfer in hollow tissues can be achieved using catheters having an electrode and/or one or more balloons. Catheters provided herein have a proximal end and a distal end, wherein the distance between the proximal and distal end corresponds to the axial length of the catheter that is selected based on the distance from the target area in the hollow tissue to outside the tissue or outside the individual. Thus, in some instances, the axial length of the catheter is from 1 cm to 200 cm. In some instances, the axial length of the catheter is from 1 cm to 100 cm (e.g., from 1 cm to 10 cm, from 10 cm to 20 cm, from 20 cm to 30 cm, from 30 cm to 40 cm, from 40 cm to 50 cm, from 50 cm to 60 cm, from 60 cm to 70 cm, from 70 cm to 80 cm, from 80 cm to 90 cm, or from 90 cm to 100 cm; e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 22 cm, about 24 cm about 25 cm, about 26 cm, about 28 cm, about 30 cm, about 32 cm, about 34 cm, about 36 cm, about 38 cm, about 40 cm, about 42 cm, about 44 cm, about 46 cm, about 48 cm, about 50 cm, about 55 cm, about 60 cm, about 65 cm, about 70 cm, about 75 cm, about 80 cm, about 85 cm, about 90 cm, about 95 cm, or about 100 cm). In particular instances in which the device is for electrotransfer in the trachea, bronchi, or bronchiole of a human individual, the axial length of the catheter is from 5 cm to 50 cm (e.g., from 5 cm to 10 cm, from 10 cm to 20 cm, from 20 cm to 30 cm, from 30 cm to 40 cm, from 40 cm to 50 cm; e.g., about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 22 cm, about 24 cm about 25 cm, about 26 cm, about 28 cm, about 30 cm, about 32 cm, about 34 cm, about 36 cm, about 38 cm, about 40 cm, about 42 cm, about 44 cm, about 46 cm, about 48 cm, or about 50 cm).

In some instances, the axial length of the catheter is from five-fold to 5,000-fold the diameter of the catheter (e.g., from 10-fold to 2,000-fold, from 50-fold to 1,000-fold, or from 100-fold to 500-fold the diameter of the catheter, e.g., from 10-fold to 20-fold, from 20-fold to 30-fold, from 30-fold to 40-fold, from 40-fold to 50-fold, from 50-fold to 60-fold, from 60-fold to 80-fold, from 80-fold to 100-fold, from 100-fold to 200-fold, from 200-fold to 500-fold, from 500-fold to 1,000-fold, from 1,000-fold to 2,000-fold, from 2,000-fold to 3,000-fold, from 3,000-fold to 4,000-fold, or from 4,000-fold to 5,000-fold the diameter of the catheter).

Catheters useful as part of the devices described herein may take various shapes, sizes, and formats. For instance, a catheter may include multiple lumens integrated within one solid structure (see, e.g., FIGS. 8A-8C). Additionally, or alternatively, a catheter has a large central lumen (wherein the geometric center of a transverse cross section of the lumen is at or near the geometric center of a transverse cross section of the catheter) and one or more (e.g., one, two, three, or more) peripheral lumens (wherein the geometric center of a transverse cross section of the lumen is peripheral (outside) a central lumen and/or within the outermost 50% of the radius of the transverse cross section of the catheter, e.g., within the outermost 40%, 35%, 30%, 25%, 20%, 15%, or 10% of the radius of the transverse cross section of the catheter).

Alternatively, the catheter may have an inner tube and an outer tube, with a space therebetween that can serve as one or more lumens (e.g., one or more inflation lumens) and/or can house one or more elongate electrodes. The space may be annular or semi-annular. For instance, in some embodiments, the inner tube has a non-circular outer diameter which, when housed in an outer tube with a circular inner diameter, creates a non-annular lumen. See, e.g., the semi-annular lumen shown in FIG. 7.

In some embodiments, the catheter has an inner diameter of 3 F to 34 F using the French scale (i.e., 1 F=⅓ mm). In some embodiments the inner diameter is 3 F, 4 F, 5 F, 6 F, 7 F, 8 F, 9 F, 10 F, 11 F, 12 F, 13 F, 14 F, 15 F, 16 F, 17 F, 18 F, 19 F, 20 F, 21 F, 22 F, 23 F, 24 F, 25 F, 26 F, 27 F, 28 F, 29 F, 30 F, 31 F, 32 F, 33 F, or 34 F. In some embodiments, the catheter has an outer diameter of 3 F to 34 F. In some embodiments the outer diameter is 3F, 4 F, 5 F, 6 F, 7 F, 8 F, 9 F, 10 F, 11 F, 12 F, 13 F, 14 F, 15 F, 16 F, 17 F, 18 F, 19 F, 20 F, 21 F, 22 F, 23 F, 24 F, 25 F, 26 F, 27 F, 28 F, 29 F, 30 F, 31 F, 32 F, 33 F, or 34 F.

In particular embodiments involving such a dual-tube system, the inner tube includes a bypass lumen and/or a delivery lumen. For instance, the bypass lumen may be the main lumen of the inner tube, and the delivery lumen may be peripheral to the bypass lumen, e.g., the delivery lumen is a smaller lumen adjacent to the bypass lumen (e.g., the main lumen). For example, in some embodiments, the geometric center of a transverse cross-section of the inner tube is within the bypass lumen, and the delivery lumen is peripheral to the geometric center. The inner tube (including both the bypass lumen and the delivery lumen) is within the outer tube. In some such instances, the geometric center of a transverse cross-section of the outer tube and the geometric center of a transverse cross-section of the inner tube are within the bypass lumen. In particular embodiments of a dual-tube catheter, an inner tube and an outer tube are concentric or near concentric (i.e., having geometric centers of a transverse cross section within 10% of the diameter of the outer tube).

In some embodiments, the catheter does not have a bypass lumen (e.g., in some devices for electrotransfer in gastrointestinal tissues or blood vessels, e.g., in which a separate bypass vessel may be employed according to known methods).

In dual-tube embodiments, the inner tube and the outer tube may be composed of different materials or the same material (e.g., polymers, such as rigid or semi-rigid polymers).

The distal end of the catheter may be fashioned as an atraumatic tip (e.g., by featuring a chamfer at or near the distal end to blunt the tip). Alternatively, the distal end of the catheter may be connected to a separate tip, which may be an atraumatic tip. Various suitable atraumatic tip designs for various tissues known in the art are contemplated herein.

The proximal end of the catheter may include or be connected to one or more inlet ports. Inlet ports may function as inlets to any of the lumens described below and may feature interface mechanisms, such as luer locks for syringes.

### Delivery Lumens and Ports

Catheters of the present invention include one or more delivery lumens. A delivery lumen runs from the proximal end of the catheter (e.g., where it may interface with an inlet port) to a delivery port proximal to the distal end of the catheter. The delivery port can be any structure for releasing an agent from the delivery lumen into contact with a hollow tissue, e.g., a target area of a hollow tissue. For instance, an agent passed distally along the catheter's axis through the delivery lumen may be redirected radially at one or more delivery ports on the catheter proximal to the distal end of the catheter, e.g., at or near an electrode. In some instances, the axial placement of the delivery port may be between the proximal end and the distal end of an electrode. In some instances, the delivery port is proximal to the electrode (e.g., within 20 mm in the axial dimension from the proximal end of the electrode, e.g., within 15 mm, within 10 mm, within 9 mm, within 8 mm, within 7 mm, within 6 mm, within 5 mm, within 4 mm, within 3 mm, within 2 mm, or within 1 mm in the axial dimension from the proximal end of the electrode). In other embodiments, the delivery port is distal to the electrode (e.g., within 20 mm in the axial dimension from the distal end of the electrode, e.g., within 15 mm, within 10 mm, within 9 mm, within 8 mm, within 7 mm, within 6 mm, within 5 mm, within 4 mm, within 3 mm, within 2 mm, or within 1 mm in the axial dimension from the distal end of the electrode). Whether the delivery port is proximal to, distal to, or within the axial length of the electrode, it may be positioned such that upon exiting the delivery port, the agent in electrical communication with the electrode and/or in contact with a target area of the hollow tissue.

In embodiments featuring a single balloon, the delivery port may be positioned on the catheter proximal to the balloon (e.g., proximal to, distal to, or within the axial length of the electrode). Such a configuration can prevent an agent exiting the delivery port from distally escaping the target area of the hollow tissue by effectively sealing the space between the outer wall of the catheter and the inner wall of the hollow tissue at or near the distal end of the target area.

In embodiments featuring a proximal balloon and a distal balloon, the delivery port may be positioned on the catheter between the balloons (i.e., proximal to the distal balloon and distal to the proximal balloon). Such a configuration can retain an agent exiting the delivery port within the target area of the hollow tissue by sealing the space between the outer wall of the catheter and the inner wall of the hollow tissue at a first point at or near the proximal end of the target area and a second point at or near the distal end of the target area.

The transverse cross-sectional shape of the delivery lumen may be circular, oval, or irregularly shaped. For instance, the transverse cross-sectional shape of the delivery lumen may be curvilinear, as shown in FIG. 11. Such shapes can be used to increase the transverse cross-sectional area of the lumen and minimize overall assembly diameter.

In some instances, the delivery port is or comprises an aerosolizer (e.g., to aerosolize a liquid composition) or nozzle (e.g., spray nozzle).

### Elongate Conductors

Devices of the invention include elongate conductors that transmit electrical energy through the catheter to a target area of the hollow tissue. A catheter of the invention may include one or more elongate conductors that run from the proximal end of the catheter to a lead proximal to the distal end of the catheter, which is the connection from the elongate conductor to the electrode. Elongate conductors can be made from any suitable conductive material, such as a metal or metal alloy. Suitable materials for elongate conductors include platinum, stainless steel, nickel, titanium, and combinations thereof, such as nitinol. In some instances, the elongate conductor is substantially the same width along the axial length of the catheter (e.g., a wire). The width (e.g., diameter) of the elongate conductor may be any size suitable to transmit electrical energy to the electrode. In some instances, the elongate conductor has a diameter of no more than 0.5 mm (e.g., no more than 0.4 mm, no more than 0.3 mm, no more than 0.25 mm, or no more than 0.2 mm, e.g., from 0.1 mm to 0.2 mm, from 0.2 mm to 0.3 mm, from 0.3 mm to 0.4 mm, or from 0.4 mm to 0.5 mm, e.g., about 0.10 mm, about 0.15 mm, about 0.20 mm, about 0.25 mm, about 0.30 mm, about 0.35 mm, about 0.40 mm, about 0.45 mm, or about 0.50 mm).

In some instances, the elongate conductor is within a tube of the catheter (a single-tube or dual-tube catheter), and the tube acts as an insulator for the elongate conductor (see, e.g., FIG. 9A). In other instances, the elongate conductor is disposed within an insulating sleeve (e.g., a polyimide sleeve) and can be within a lumen of the catheter (e.g., within an inflation lumen, or within a lumen created by an outer or inner tube of a dual-tube catheter.

### Inflation Lumens and Ports

The invention contemplates several arrangements of inflation lumens, depending on the number of balloons and whether the balloons are independently or simultaneously inflatable. In some instances of single-balloon devices, one or more inflation lumens runs from the proximal end of the catheter to an inflation port proximal to the distal end of the catheter. The inflation port is positioned such that an inflation medium (e.g., air) exiting the inflation lumen through the inflation port inflates the balloon (e.g., distal balloon). Thus, the inflation port may be covered by the balloon.

In some embodiments, the inflation lumen is provided by an inflation tube, which may be a peripheral tube disposed on (e.g., bundled against) the outside of a central tube (e.g., a bypass tube) (see, e.g., FIG. 2). The transverse cross-sectional shape of an inflation lumen may be circular, oval, or irregularly shaped. For instance, the transverse cross-sectional shape of the inflation lumen may be curvilinear, as shown in FIG. 11. Such shapes can be used to increase the transverse cross-sectional area of the lumen and minimize overall assembly diameter.

In some instances of dual-balloon devices, there are two separate (independent) inflation lumens (see, e.g., FIG. 9A). A proximal inflation lumen runs to a proximal inflation port to inflate a proximal balloon, and a distal inflation lumen runs to a distal inflation port to inflate a distal balloon.

In other instances of dual-balloon devices, a single inflation lumen inflates both proximal and distal balloons. Inflation of the balloons by a single inflation lumen may occur simultaneously or near simultaneously, depending on whether the external forces are similar on each balloon.

### Bypass Lumens

Some embodiments of the catheters described herein feature a bypass lumen configured to allow passage of material (e.g., airflow) across the entire axial length of the catheter (from the proximal end of the catheter to beyond the distal end of the catheter and/or from beyond the distal end of the catheter to the proximal end of the catheter). A bypass lumen may be included in any of the catheters described herein to allow the catheter to be positioned in a tubular tissue without occluding flow through the tissue. For instance, in devices configured for airway electrotransfer, a bypass lumen may function to allow an individual to breath while the device is in place by allowing airflow through the axial length of the catheter. Such bypass lumens may run from the proximal end of the catheter to the distal end of the catheter, but it will be understood that other configurations are available that allow passage of material across the catheter. For instance, a bypass lumen may terminate at a port proximal to the distal end of the catheter or distal to the proximal end of the catheter.

A bypass lumen (or a plurality of bypass lumens, collectively) may occupy any suitable proportion of a catheter's transverse cross sectional area, and such proportion will be influenced by factors such as required flow rate of material through the bypass lumen, width (e.g., diameter) of the catheter, and mechanical stability of the catheter material (e.g., to withstand bending and compressive forces as the operator moves the catheter through a hollow tissue). In some instances, the bypass lumen occupies 10% to 95% of the catheter's transverse cross-sectional area (e.g., 15% to 90%, 20% to 85%. 25% to 80%, or 30% to 70% of the catheter's transverse cross-sectional area, e.g., 10% to 20%, 20% to 30%, 30% to 40%, 40% to 50%, 50% to 60%, 60% to 70%, 70% to 80%, or 80% to 90% of the catheter's transverse cross-sectional area).

In any of the aforementioned embodiments, the bypass lumen may have a transverse cross sectional area from 2 mm² to 250 mm² (e.g., from 5 mm² to 200 mm², from 10 mm² to 150 mm², or from 20 mm² to 100 mm²; e.g., from 2 mm² to 4 mm², from 4 mm² to 6 mm², from 6 mm² to 8 mm², from 8 mm² to 10 mm², from 10 mm² to 12 mm², from 12 mm² to 15 mm², from 15 mm² to 20 mm², from 20 mm² to 25 mm², from 25 mm² to 30 mm², from 30 mm² to 40 mm², from 40 mm² to 50 mm², from 50 mm² to 60 mm², from 60 mm² to 70 mm², from 70 mm² to 80 mm², from 80 mm² to 90 mm², from 90 mm² to 100 mm², from 100 mm² to 125 mm², from 125 mm² to 150 mm², from 150 mm² to 200 mm², or from 200 mm² to 250 mm²). The transverse cross-sectional shape of the bypass lumen may be circular, oval, or irregularly shaped.

### Balloons

Devices of the invention include one or more balloons disposed about the catheter (e.g., covering the perimeter of the catheter such that inflation occurs radially). Various balloons are known in the art for occluding biological passageways, such as blood vessels, and may be readily adapted for use as part of the present devices and methods. In particular embodiments of the present invention, the balloon is a compliant (stretch) balloon. Compliant balloons can be made from a variety of elastic materials, such as polyurethane, thermoplastic elastomers, styrenic block copolymers, or isoprene rubber. Alternatively, non-compliant balloons may be used, e.g., pebax material. A balloon may be in the form of a thin-walled cylinder in its relaxed state and attached at its proximal and distal ends to the catheter to form an airtight seal. It is advantageous to attach a balloon to the catheter in a manner that minimizes changes in the outer diameter of the catheter. Thus, in some instances, the outer diameter of the deflated balloon is substantially flush with the outer diameter of the catheter. This can be achieved by fabricating a step down in the outer surface of the catheter to accommodate the thickness of the terminus of the tube to be attached.

In single-balloon devices, the single balloon is disposed about the catheter over the inflation port (e.g., distal inflation port) distal to the delivery port and the electrode, thereby confining the agent to the target area proximal to the balloon by forming a seal with the hollow tissue.

In dual-balloon devices, the distal balloon is disposed about the catheter over the distal inflation port distal to the delivery port and the electrode, and the proximal balloon is disposed about the catheter over the proximal inflation port proximal to the delivery port and the electrode, thereby confining the agent to the target area between the proximal and distal balloons by forming seals with the hollow tissue. The proximal balloon and the distal balloon may be substantially the same size and/or material. In other embodiments, the proximal balloon and the distal balloon are different (e.g., the proximal balloon is larger than the distal balloon, or the distal balloon is larger than the distal balloon).

The axial length of each of the one or more balloons may be from one-fold to 50-fold the outer diameter of the catheter (e.g., from two-fold to 30-fold, from five-fold to 20-fold, or from eight-fold to 12-fold the outer diameter of the catheter, e.g., from one-fold to two-fold, from two-fold to three-fold, from three-fold to four-fold, from four-fold to five-fold, from five-fold to six-fold, from six-fold to seven-fold, from seven-fold to eight-fold, from eight-fold to 10-fold, from 10-fold to 15-fold, from 15-fold to 20-fold, from 20-fold to 30-fold, from 30-fold to 40-fold, or from 40-fold to 50-fold the outer diameter of the catheter).

In some instances, the balloon is radially inflatable to a diameter from 20% to 500% greater than the outer diameter of the balloon in its relaxed shape (e.g., from 30% to 400%, from 40% to 300%, from 50% to 200%, or from 75% to 150% greater than the outer diameter of the balloon in its relaxed shape, e.g., from 20% to 30%, from 30% to 40%, from 40% to 50%, from 50% to 60%, from 60% to 75%, from 75% to 100%, from 100% to 120% from 120% to 150%, from 150% to 200%, from 200% to 250%, from 250% to 300%, from 300% to 400%, or from 400% to 500% greater than the outer diameter of the balloon in its relaxed shape).

Balloons may include other elements as desirable for each application. For instance, a balloon may include a pressure sensor to indicate when the balloon is sufficiently inflated. Such pressure sensors may detect the force of the balloon against a wall of the hollow tissue. Additionally, or alternatively, the balloon may be lubricated or otherwise treated, e.g., to reduce or prevent adhesion to the hollow tissue after inflation. Other balloon elements known in the art can be applied according to known methods.

### Electrodes

The invention includes devices having electrodes configured to transmit electrical energy to hollow tissues. An electrode is disposed about the catheter, e.g., proximal to a balloon. Generally, the electrode may be wrapped around the circumference of the catheter such that current transmitted from the catheter flows radially from the catheter and/or the electrode. The electrode may be a conductive mesh, a spiral (e.g., a wire spiral), or a combination thereof (e.g., a wire spiral connected to a conductive mesh). The electrode can be made from any suitable conductive material, such as a metal or metal alloy. Suitable materials for electrodes include platinum, stainless steel, nickel, titanium, and combinations thereof, such as nitinol or a platinum/iridium alloy.

In some embodiments, the axial length of the electrode is from two-fold to 100-fold the outer diameter of the catheter (e.g., from three-fold to 90-fold, from four-fold to 80-fold, from five-fold to 50-fold, from six-fold to 30-fold, from eight-fold to 20-fold, or from 10-fold to 15-fold the outer diameter of the catheter, e.g., from two-fold to ten-fold, from ten-fold to 50-fold, or from 50-fold to 100-fold the outer diameter of the catheter, e.g., from two-fold to three-fold, from three-fold to four-fold, from four-fold to five-fold, from five-fold to six-fold, from seven-fold to eight-fold, from eight-fold to nine-fold, from nine-fold to 10-fold, from 10-fold to 12-fold, from 12-fold to 15-fold, from 15-fold to 20-fold, from 20-fold to 30-fold, from 30-fold to 40-fold, from 40-fold to 50-fold, from 50-fold to 60-fold, from 60-fold to 70-fold, from 70-fold to 80-fold, from 80-fold, from 80-fold to 90-fold, or from 90-fold to 100-fold the outer diameter of the catheter).

In some instances, the electrode is divided into multiple, electrically independent segments (e.g., two, three, four, five, or more electrically independent segments). Each segment can transmit electrical energy at different times so as to maximize the target area of the hollow tissue while reducing the total current, compared to the current resulting from an electrode of the same size without being electrically segmented.

The electrode (or each independent segment of an electrode) is connected to the elongate conductor at a lead (i.e., the point of attachment of the elongate conductor to the electrode). The lead may be a separate element (e.g., a wire) or simply the interface of the elongate conductor with the electrode).

### III. Systems

The invention includes systems involving any of the aforementioned devices. A system may include one or more additional devices or elements. In some instances, a system of the invention includes any of the devices described herein in which the elongate conductor is connected to a waveform generator and/or power supply according to methods generally known in the art. Additionally, or alternatively, a system may include the device in combination with one or more actuators, such as syringes, for inflation of the one or more balloons or administration of the agent.

### IV. Methods

Featured herein are methods of using any of the devices described herein. Such methods include methods of exposing a target area in a hollow tissue to electrical energy, methods of delivering an agent to a target cell, methods of expressing a nucleic acid vector in a target cell in a hollow tissue, and methods of treating a disease or disorder in an individual.

Hollow tissues amenable to electrotransfer using the methods of the invention include tubular or luminal tissues or organs, such as an airway (e.g., trachea, larynx, bronchus (e.g., main bronchus), or bronchiole), blood vessel (e.g., artery or vein), or gastrointestinal tract tissue (e.g., esophagus, stomach, duodenum, jejunum, colon, or rectum). Target cells include epithelial cells of the hollow tissue. Target cells in the airway include cells of the target area, including tracheal cells, bronchial cells, and bronchiolar cells (e.g., tracheal epithelial cells, bronchial epithelial cells, and bronchiolar epithelial cells). In some instances, the target cell is a Club cell.

In some instances, provided are methods of exposing a target area in a hollow tissue (e.g., a region of an airway, such as a region of trachea, bronchus, or bronchiole) to electrical energy (e.g., voltage and/or current) using a single-balloon device of the invention. An operator (e.g., technician, physician, or other medical professional) handling a single-balloon device as described herein positions the electrode within the hollow tissue such that the region of the hollow tissue radially adjacent to the electrode includes all or a portion of the target area (the area to be exposed to the electrical energy). Next, an operator flows an inflation medium (e.g., air, other gas, or liquid) distally through the inflation lumen to inflate the balloon such that the balloon contacts the hollow tissue. The balloon may contact the hollow tissue to the extent necessary to effectively seal off the space between the outer wall of the catheter and inner wall of the hollow tissue proximal to the balloon, e.g., to substantially prevent leakage of liquid pharmaceutical composition from the target area proximal to the balloon. Once the balloon is inflated and the device is in position, an operator introduces an agent (e.g., a therapeutic agent, e.g., a naked nucleic acid), or a pharmaceutical composition thereof, through the delivery lumen such that it exits the catheter at the delivery port and contacts the target area. While the balloon is still inflated, and after introducing the agent to the target area, electrical energy is transmitted through the electrode, thereby exposing the target area to the electrical energy. The electrical energy may be transmitted at conditions suitable for electrotransfer of the agent into a target cell in the target area. After electrical energy transmission, the balloon can be deflated, and the device can be removed.

Additionally, or alternatively, the present methods can be used to deliver an agent (e.g., a therapeutic agent, e.g., a naked nucleic acid) to target area or a target cell in a hollow tissue (e.g., a region of an airway, such as a region of trachea, bronchus, or bronchiole). An operator (e.g., technician, physician, or other medical professional) handling a single-balloon device as described herein positions the electrode within the hollow tissue such that the region of the hollow tissue radially adjacent to the electrode includes all or a portion of the target area (the area to be exposed to the electrical energy). Next, an operator flows an inflation medium (e.g., air, other gas, or liquid) distally through the inflation lumen to inflate the balloon such that the balloon contacts the hollow tissue. The balloon may contact the hollow tissue to the extent necessary to effectively seal off the space between the outer wall of the catheter and inner wall of the hollow tissue proximal to the balloon, e.g., to substantially prevent leakage of liquid pharmaceutical composition from the target area proximal to the balloon. Once the balloon is inflated and the device is in position, an operator introduces the agent (e.g., a therapeutic agent, e.g., a naked nucleic acid), or a pharmaceutical composition thereof, through the delivery lumen such that it exits the catheter at the delivery port and contacts the target area. While the balloon is still inflated, and after introducing the agent to the target area, electrical energy is transmitted through the electrode at conditions suitable for electrotransfer of the agent into the target cell in the target area. After electrical energy transmission, the balloon can be deflated, and the device can be removed. In some instances, in which the agent is a nucleic acid vector, the nucleic acid vector may be expressed in a target cell, and/or the method may include expressing and/or detecting expression of the nucleic acid vector in the target area or elsewhere in the individual. Any suitable methods of detection are contemplated herein, including detection of a nucleic acid (e.g., an RNA transcript) or protein expressed from the nucleic acid vector.

In some instances, any of the methods for electrotransfer in an airway may include partial airway epithelial ablation therapy, e.g., to prime the tissue for electrotransfer. For instance, partial epithelial ablation therapy can be used to remove all or a portion of a mucus layer (e.g., mucus and/or columnar cells), thereby exposing target cells (e.g., target cells in the trachea or bronchi (e.g., basal stem cells and or goblet cells)). Partial epithelial ablation therapy can be carried out using a clinically available transbronchoscopic pulsed electric field system. Alternatively, any of the methods for electrotransfer in an airway may be performed in an individual after partial airway epithelial ablation therapy (e.g., as described herein) in the individual. Diseases or disorders treatable using such partial airway epithelial ablation therapy schemes include chronic inflammatory lung diseases or disorders (e.g., chronic obstructive pulmonary disease).

Methods described herein can be used for treating an individual, e.g., a human. In some embodiments, the individual has been diagnosed with a disease or disorder (e.g., a monogenic disease, e.g., a monogenic respiratory disease). In particular instances, the method includes delivering an agent configured to treat the disease or disorder (e.g., approved for the treatment of the disease or disorder). For instance, the agent delivered by the methods of the invention may include a therapeutic gene or sequence that replaces a defective protein associated with the disease or disorder (e.g., CFTR for the treatment of cystic fibrosis).

In some embodiments of methods for treating a respiratory disease or disorder (in which the electrode is positioned within an airway, e.g., trachea, bronchus, or bronchiole), the individual has (e.g., and is being treated for) cystic fibrosis, primary ciliary dyskinesia, congenital alveolar proteinosis, or pulmonary alveolar microlithiasis. In some embodiments, the individual has (e.g., and is being treated for) a chronic inflammatory lung disease or disorder (e.g., chronic obstructive pulmonary disease, asthma, chronic rhinosinusitis, emphysema, fibrosis, or pneumonia).

The aforementioned methods of using a single-balloon device can be used to treat a disease or disorder in an individual (e.g., a disease or disorder associated with a therapeutic sequence encoded by a nucleic acid vector delivered to the individual). Methods of treatment include introducing an effective amount of the agent (e.g., a therapeutic agent) through the delivery lumen such that the agent contacts the region of hollow tissue radially adjacent to the electrode.

In any of the preceding methods involving single-tube devices (e.g., where the hollow tissue is an airway), an individual may be positioned such that the distal end of the catheter is below the proximal end to allow gravity to bring the agent, or pharmaceutical composition thereof, against the balloon. For example, an individual may be seated in an upright position (e.g., partial or full Fowler's position).

In other embodiments, the methods involve exposing a target area in a hollow tissue (e.g., a region of an airway, such as a region of trachea, bronchus, or bronchiole) to electrical energy (e.g., voltage and/or current) using a dual-balloon device of the invention. An operator (e.g., technician, physician, or other medical professional) handling a dual-balloon device as described herein positions the electrode within the hollow tissue such that the region of the hollow tissue radially adjacent to the electrode includes all or a portion of the target area (the area to be exposed to the electrical energy). Next, an operator flows an inflation medium (e.g., air, other gas, or liquid) distally through the one or more (e.g., one or both) inflation lumen(s) to inflate the balloon such that the balloon contacts the hollow tissue. In a catheter having two inflation lumens, inflation may be performed simultaneously for both balloons or independently (e.g., the distal balloon may be inflated before the proximal balloon, e.g., the distal balloon may be inflated prior to introducing the agent and the proximal balloon may be inflated after introducing the agent, or vice versa). The balloons may contact the hollow tissue to the extent necessary to effectively seal off the space between the outer wall of the catheter and inner wall of the hollow tissue proximal to the distal balloon and distal to the proximal balloon, e.g., to substantially prevent leakage of liquid pharmaceutical composition from the target area distal to the proximal balloon and proximal to the distal balloon. Once the balloons are inflated and the device is in position, an operator introduces an agent (e.g., a therapeutic agent, e.g., a naked nucleic acid), or a pharmaceutical composition thereof, through the delivery lumen such that it exits the catheter at the delivery port and contacts the target area. While the balloons are still inflated, and after introducing the agent to the target area, electrical energy is transmitted through the electrode, thereby exposing the target area to the electrical energy. The electrical energy may be transmitted at conditions suitable for electrotransfer of the agent into a target cell in the target area. After electrical energy transmission, the balloons can be deflated, and the device can be removed.

Additionally, or alternatively, the present methods can be used to deliver an agent (e.g., a therapeutic agent, e.g., a naked nucleic acid) to target area or a target cell in a hollow tissue (e.g., a region of an airway, such as a region of trachea, bronchus, or bronchiole). An operator (e.g., technician, physician, or other medical professional) handling a dual-balloon device as described herein positions the electrode within the hollow tissue such that the region of the hollow tissue radially adjacent to the electrode includes all or a portion of the target area (the area to be exposed to the electrical energy). Next, an operator flows an inflation medium (e.g., air, other gas, or liquid) distally through the one or more inflation lumens to inflate the balloons such that the balloon contacts the hollow tissue. In a catheter having two inflation lumens, inflation may be performed simultaneously for both balloons or independently (e.g., the distal balloon may be inflated before the proximal balloon, e.g., the distal balloon may be inflated prior to introducing the agent and the proximal balloon may be inflated after introducing the agent, or vice versa). The balloons may contact the hollow tissue to the extent necessary to effectively seal off the space between the outer wall of the catheter and inner wall of the hollow tissue proximal to the distal balloon and distal to the proximal balloon, e.g., to substantially prevent leakage of liquid pharmaceutical composition from the target area distal to the proximal balloon and proximal to the distal balloon. Once the balloon is inflated and the device is in position, an operator introduces the agent (e.g., a therapeutic agent, e.g., a naked nucleic acid), or a pharmaceutical composition thereof, through the delivery lumen such that it exits the catheter at the delivery port and contacts the target area. While the balloons are still inflated, and after introducing the agent to the target area, electrical energy is transmitted through the electrode at conditions suitable for electrotransfer of the agent into the target cell in the target area. After electrical energy transmission, the balloons can be deflated, and the device can be removed. In some instances, in which the agent is a nucleic acid vector, the nucleic acid vector may be expressed in a target cell, and/or the method may include expressing and/or detecting expression of the nucleic acid vector in the target area or elsewhere in the individual. Any suitable methods of detection are contemplated herein, including detection of a nucleic acid (e.g., an RNA transcript) or protein expressed from the nucleic acid vector.

The aforementioned methods of using a dual-balloon device can be used to treat a disease or disorder in an individual (e.g., a disease or disorder associated with a therapeutic sequence encoded by a nucleic acid vector delivered to the individual). Methods of treatment include introducing an effective amount of the agent (e.g., a therapeutic agent) through the delivery lumen such that the agent contacts the region of hollow tissue radially adjacent to the electrode.

In any of the preceding methods involving dual-tube devices (e.g., wherein the hollow tissue is an airway), an individual need not be positioned such that the distal end of the catheter is below the proximal end to allow gravity to bring the agent, or pharmaceutical composition thereof, against the balloon. For example, in some embodiments, it is not necessary to position the individual in an upright position (e.g., partial or full Fowler's position). Rather, the individual (and/or the hollow tissue, e.g., tubular tissue) may be horizontally positioned, e.g., supine.

Conditions suitable for electrotransfer of an agent (e.g., a therapeutic agent, e.g., a naked nucleic acid) into a target cell in a target area comprise a voltage at the target area or target cell from 10 V to 5,000 V (e.g., from 20 V to 2,500 V, from 30 V to 2,000 V, from 50 V to 1,500 V, from 100 V to 1,000 V, or from 200 V to 500 V, e.g., from 10 V to 20 V, from 20 V to 30 V, from 30 V to 50 V, from 50 V to 100 V, from 100 V to 200 V, from 200 V to 500 V, from 500 V to 1,000 V, from 1,000 V to 1,500 V, from 1,500 V to 2,000 V, from 2,000 V to 2,500 V, from 2,500 V to 3,000 V, from 3,000 V to 4,000 V, or from 4,000 V to 5,000 V). Conditions suitable for electrotransfer of an agent (e.g., a therapeutic agent, e.g., a naked nucleic acid) into a target airway cell comprise a voltage at the target lung cell of less than 2,400 V. In some embodiments, the voltage at the target airway cell is between 10 V and 2,400 V, e.g., between 100 V and 2,400 V, e.g., from 100 V to 500 V, from 500 V to 1,000 V, from 1,000 V to 1,500 V, or from 1,500 V to 2,000 V. Such voltages can be achieved by administering one or more, e.g., 1-10 (e.g., 1-6) pulses of electrical energy. In some embodiments, the one or more pulses of electrical energy have an amplitude from 10 V to 10,000 V, e.g., from 100 V to 5,000 V, e.g., from 100 V to 500 V, from 500 V to 1,000 V, from 1,000 V to 1,500 V, or from 1,500 V to 2,000 V. In some embodiments, each of the pulses of electrical energy is from 0.01 to 200 milliseconds in duration. In some embodiments, each of the pulses of electrical energy is from 10 to 50 milliseconds in duration (e.g., about 20 milliseconds in duration). In some embodiments, the total duration of all of the pulses of electrical energy is from 10 to 50 milliseconds (e.g., about 20 milliseconds in total duration). In some embodiments, the electrode is a monopolar electrode. In other embodiments, the electrode is part of a bipolar electrode configuration.

In some embodiments, the total number of pulses of electrical energy are transmitted within 1-20 seconds. In some embodiments, a single pulse (i.e., one and only one pulse) of electrical energy is transmitted. In other embodiments, exactly two pulses of electrical energy are transmitted. In some embodiments, the one or more pulses of electrical energy are square waveforms.

In some embodiments, the electrode is within 10 cm of the target cell. For example, in some embodiments, the electrode is within 1 cm of the target cell (e.g., within 5 mm, within 4 mm, or within 3 mm of the target cell).

An agent can be administered within 24 hours of transmission of an electric field (e.g., within 20 hours, within 18 hours, within 16 hours, within 14 hours, within 12 hours, within 10 hours, within 8 hours, within 6 hours, within 4 hours, within 3 hours, within 2 hours, within 90 minutes, within 60 minutes, within 45 minutes, within 30 minutes, within 20 minutes, within 15 minutes, within 10 minutes, within 5 minutes, within 4 minutes, within 3 minutes, within 2 minutes, within 1 minute, within 45 seconds, within 30 seconds, within 20 seconds, within 15 seconds, within 10 seconds, or within 5 seconds preceding transmission of an electric field).

In any of the preceding methods, the hollow tissue may be a tubular tissue, such as an airway (e.g., trachea, bronchus, and/or bronchiole), a gastrointestinal tract tissue, or a blood vessel. In some instances, a target tissue is an epithelial tissue (e.g., an airway epithelium). For instance, a target cell in a target tissue may be an epithelial cell (e.g., a basal epithelial cell). In some instances, the target cell is a resident stem cell. Alternatively, the target cell may be a terminally differentiated cell (e.g., a terminally differentiated epithelial cell). In some instances, the target cell is separated from the inner wall surface of the hollow tissue by one or more layers of cells or mucous.

As a general proposition, an effective amount of the therapeutic agent, e.g., nucleic acid vector (e.g., circular DNA vector) administered to the individual (e.g., human) may be in the range from 1.0 pg to 1 mg of nucleic acid (e.g., from 0.01 ng to 100 μg, from 0.1 ng to 50 μg, from 1 ng to 10 μg, or from 10 ng to 1 μg, e.g., from 0.01 ng to 0.05 ng, from 0.05 ng to 0.1 ng, from 0.1 ng to 0.5 ng, from 0.5 ng to 1 ng, from 1 ng to 5 ng, from 5 ng to 10 ng, from 10 ng to 50 ng, from 50 ng to 100 ng, from 100 ng to 500 ng, from 500 ng to 1 μg, from 1 μg to 5 μg, or from 5 μg to 10 μg, e.g., about 1 pg, about 5 pg, about 10 pg, about 20 pg, about 25 pg, about 50 pg, about 75 pg, about 100 pg, about 1 ng, about 5 ng, about 10 ng, about 20 ng, about 25 ng, about 50 ng, about 75 ng, about 100 ng, about 1 μg, about 2 μg, about 3 μg, about 4 μg, about 5 μg, about 6 μg, about 7 μg, about 8 μg, about 9 μg, about 10 μg, about 15 μg, about 20 μg, about 25 μg, about 50 μg, about 75 μg, about 100 μg, or about 500 μg).

Dosages for a therapeutic agent (e.g., nucleic acid vector (e.g., circular DNA vector)) or pharmaceutical composition thereof as described herein may be determined empirically in individuals who have been given one or more administrations of the nucleic acid vector (e.g., circular DNA vector) or pharmaceutical composition thereof. Individuals may be given incremental dosages of the therapeutic agent, e.g., nucleic acid vector (e.g., circular DNA vector) or pharmaceutical composition thereof. To assess efficacy of the nucleic acid vector (e.g., circular DNA vector) or pharmaceutical composition thereof, an indicator of the disease/disorder can be monitored. For repeated administrations over several days or longer, depending on the condition, the treatment is sustained until a desired result in the diseased or disorder is achieved.

In some instances, a single injection of the therapeutic agent, e.g., nucleic acid vector (e.g., circular DNA vector) or pharmaceutical composition thereof is administered to the individual over the course of the treatment. In some embodiments, the single injection of the therapeutic agent, e.g., nucleic acid vector (e.g., circular DNA vector) or pharmaceutical composition thereof is administered to the individual.

The therapeutic agent, e.g., nucleic acid vector (e.g., circular DNA vector) or pharmaceutical composition thereof can be administered as a single dose. Alternatively, the therapeutic agent, e.g., nucleic acid vector (e.g., circular DNA vector) or pharmaceutical composition thereof can be administered in multiple doses (e.g., two or more doses, three or more doses, four or more doses, five or more doses, six or more doses, e.g., two doses, three doses, four doses, five doses, or six doses) over the course of a treatment.

In some embodiments, dosing frequency is once per day, once every other day, three times per week, or twice per week. In some embodiments, dosing frequency is once every week, once every two weeks, once every three weeks, once every four weeks, once every five weeks, once every six weeks, once every seven weeks, once every eight weeks, once every nine weeks, or once every ten weeks; or once every month, once every two months, or once every three months, or less frequently. The progress of therapy can be readily monitored (e.g., detected or quantified), according to methods known in the art and described herein. The dosing regimen of the therapeutic agent, e.g., nucleic acid vector (e.g., circular DNA vector) or pharmaceutical composition thereof can vary over time.

### Agents and Compositions

Agents for use with the devices, systems, and methods of the present invention include therapeutic agents (e.g., nucleic acid vectors encoding therapeutic sequences or genes (e.g., DNA vectors or RNA vectors, e.g., self-replicating RNA vectors), therapeutic nucleic acid vectors (e.g., miRNAs), small molecules, and biologics (e.g., therapeutic proteins))) and non-therapeutic agents (e.g., reporter gene vectors). In some instances, in which an individual is being treated, the agent is a therapeutic agent.

Agents amenable to electrotransfer include non-viral vectors, e.g., naked nucleic acid vectors. Naked nucleic acid vectors include DNA and RNA. Naked DNA vectors and naked RNA vectors may be circular (e.g., plasmid DNA vectors or synthetic circular DNA vectors). In particular instances, the agent (e.g., therapeutic agent) is a synthetic circular DNA vector.

Synthetic circular DNA vectors persist intracellularly (e.g., in dividing or in quiescent cells, such as post-mitotic cells) as episomes, e.g., in a manner similar to AAV vectors. In any of the embodiments, described herein, a synthetic circular DNA vector may be a non-integrating vector. Synthetic circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG islands or CpG motifs)) or components additionally, or otherwise associated with reduced persistence (e.g., CpG islands or CpG motifs). The synthetic circular DNA vectors produced as described herein feature one or more therapeutic sequences and may lack plasmid backbone elements (e.g., bacterial elements such as (i) a bacterial origin of replication and/or (ii) a drug resistance gene) and a recombination site.

Naked circular DNA vectors are devoid of components inherent to viral vectors (e.g., viral proteins) and bacterial plasmid DNA, such as immunogenic components (e.g., immunogenic bacterial signatures (e.g., CpG motifs)) or components additionally or otherwise associated with reduced persistence (e.g., CpG islands). For example, in some embodiments, the vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dem methylation. For examples, in some embodiments, the vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dem methylase).

In some embodiments, the synthetic circular DNA vector is persistent in vivo (e.g., the therapeutic circular DNA vector exhibits improved expression persistence (e.g., intra-cellular persistence and/or trans-generational persistence) and/or therapeutic persistence relative to a reference vector, e.g., a circular DNA vector produced in bacteria or having one or more bacterial signatures that are not present in the vector of the invention, e.g., plasmid DNA). In some embodiments, expression persistence of the synthetic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, intra-cellular persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, therapeutic persistence of the therapeutic circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, the reference vector is a circular vector or plasmid that (a) has the same therapeutic sequence as a therapeutic circular DNA vector to which it is being compared, and (b) is produced in bacteria and/or has one or more bacterial signatures that are not present in the therapeutic circular DNA vector to which it is being compared, which signatures may include, for example, an antibiotic resistance gene or a bacterial origin of replication.

In some embodiments, expression of a synthetic circular DNA vector persists for one week, two weeks, three weeks, four weeks, six weeks, two months, three months, four months, five months, six months, seven months, eight months, nine months, ten months, eleven months, one year, or longer after administration.

In some embodiments, expression and/or therapeutic effect of the synthetic circular DNA vector persists for one week to four weeks, from one month to four months, or from four months to one year (e.g., at least one week, at least two weeks, at least one month, or longer). In some embodiments, the expression level of the synthetic circular DNA vector does not decrease by more than 90%, by more than 50%, or by more than 10% in the 1 week or more, e.g., 2 weeks, 3 weeks, 5 weeks, 7 weeks, 9 weeks or more, 13 weeks or more, 18 weeks or more following transfection from levels observed within the first 1, 2, or 3 days.

The synthetic circular DNA vector may be monomeric, dimeric, trimeric, tetrameric, pentameric, hexameric, etc. In some preferred embodiments, the circular DNA vector is monomeric. In some embodiments, the DNA vector is supercoiled. In some embodiments, the therapeutic circular DNA vector is a monomeric, supercoiled circular DNA molecule. In some embodiments, the therapeutic circular DNA vector is nicked. In some embodiments, the therapeutic circular DNA vector is open circular. In some embodiments, the therapeutic circular DNA vector is double-stranded circular.

Synthetic circular DNA vectors described herein contain a therapeutic sequence, which may include one or more protein-coding domain and/or one or more non-protein coding domains (e.g., a therapeutic nucleic acid).

In particular embodiments involving a therapeutic protein-coding therapeutic domain, the therapeutic sequence includes, linked in the 5′ to 3′ direction: a promoter and a single therapeutic protein-coding domain (e.g., a single transcription unit); a promoter and two or more therapeutic protein-coding domains (e.g., a multicistronic unit); or a first transcription unit and one or more additional transcription units (e.g., a multi-transcription unit). Any such protein-coding therapeutic sequences may further include non-protein coding domains, such as polyadenylation sites, control elements, enhancers, sequences to mark DNA (e.g., for antibody recognition), PCR amplification sites, sequences that define restriction enzyme sites, site-specific recombinase recognition sites, sequences that are recognized by a protein that binds to and/or modifies nucleic acids, linkers, splice sites, pre-mRNA binding domains, regulatory sequences, and/or a therapeutic nucleic acid (e.g., a microRNA-encoding sequence). Therapeutic protein-coding domains can be full-length protein-coding domains (e.g., corresponding to a native gene or natural variant thereof) or a functional portion thereof, such as a truncated protein-coding domain (e.g., minigene).

In some embodiments, the therapeutic sequence encodes a monomeric protein (e.g., a monomeric protein with secondary structure under physiological conditions, e.g., a monomeric protein with secondary and tertiary structure under physiological conditions, e.g., a monomeric protein with secondary, tertiary, and quaternary structure under physiological conditions). Additionally, or alternatively, the therapeutic sequence may encode a multimeric protein (e.g., a dimeric protein (e.g., a homodimeric protein or heterodimeric protein), a trimeric protein, etc.)

In some embodiments, the therapeutic sequence encodes a therapeutic protein associated with treating a disease (e.g., the disease to be treated by any of the present methods of treatment). In instances in which the disease or disorder is a respiratory disease or disorder (e.g., cystic fibrosis, primary ciliary dyskinesia, congenital alveolar proteinosis, pulmonary alveolar microlithiasis, chronic obstructive pulmonary disease, asthma, chronic rhinosinusitis, emphysema, fibrosis, or pneumonia), the therapeutic sequence encodes a therapeutic protein associated with treating a respiratory disorder. In some embodiments (e.g., in which the individual is being treated for cystic fibrosis), the therapeutic sequence encodes cystic fibrosis transmembrane regulator (CFTR).

In some embodiments, the therapeutic sequence is from 0.1 Kb to 100 Kb in length (e.g., the therapeutic gene sequence is from 0.2 Kb to 90 Kb, from 0.5 Kb to 80 Kb, from 1.0 Kb to 70 Kb, from 1.5 Kb to 60 Kb, from 2.0 Kb to 50 Kb, from 2.5 Kb to 45 Kb, from 3.0 Kb to 40 Kb, from 3.5 Kb to 35 Kb, from 4.0 Kb to 30 Kb, from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 0.1 Kb to 0.5 Kb, from 0.5 Kb to 1.0 Kb, from 1.0 Kb to 2.5 Kb, from 2.5 Kb to 4.5 Kb, from 4.5 Kb to 8 Kb, from 8 Kb to 10 Kb, from 10 Kb to 15 Kb, from 15 Kb to 20 Kb in length, or greater, e.g., from 0.1 Kb to 0.25 Kb, from 0.25 Kb to 0.5 Kb, from 0.5 Kb to 1.0 Kb, from 1.0 Kb to 1.5 Kb, from 1.5 Kb to 2.0 Kb, from 2.0 Kb to 2.5 Kb, from 2.5 Kb to 3.0 Kb, from 3.0 Kb to 3.5 Kb, from 3.5 Kb to 4.0 Kb, from 4.0 Kb to 4.5 Kb, from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater). In some embodiments, the therapeutic sequence is at least 10 Kb (e.g., from 10 Kb to 15 Kb, from 15 Kb to 20 Kb, or from 20 Kb to 30 Kb; e.g., from 10 Kb to 13 Kb, from 10 Kb to 12 Kb, or from 10 Kb to 11 Kb; e.g., from 10-11 Kb, from 11-12 Kb, from 12-13 Kb, from 13-14 Kb, or from 14-15 Kb). In some embodiments, the therapeutic sequence is at least 1,100 bp in length (e.g., from 1,100 bp to 10,000 bp, from 1,100 bp to 8,000 bp, or from 1,100 bp to 5,000 bp in length). In some embodiments, the therapeutic sequence is at least 2,500 bp in length (e.g., from 2,500 bp to 15,000 bp, from 2,500 bp to 10,000 bp, or from 2,500 bp to 5,000 bp in length, e.g., from 2,500 bp to 5,000 bp, from 5,000 bp to 7,500 bp, from 7,500 bp to 10,000 bp, from 10,000 bp to 12,500 bp, or from 12,500 bp to 15,000 bp). In some embodiments, the therapeutic sequence is at least 8,000 bp, at least 9,000 bp, at least 10,000 bp, at least 11,000 bp, at least 12,000 bp at least 13,000 bp, at least 14,000 bp, at least 15,000 bp, at least 16,000 bp (e.g., 11,000 bp to 16,000 bp, 12,000 bp to 16,000 bp, 13,000 bp to 16,000 bp, 14,000 bp to 16,000 bp, or 15,000 bp to 16,000 bp). In particular embodiments, the therapeutic sequence is sufficiently large to encode a protein and is not an oligonucleotide therapy (e.g., not an antisense, siRNA, shRNA therapy, etc.).

In some embodiments, the 3′ end of the therapeutic sequence is connected to the 5′ end of the therapeutic sequence in a therapeutic circular DNA vector by a non-bacterial sequence of no more than 30 bp (e.g., from 3 bp to 24 bp, from 4 bp to 18 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, or from 24 bp to 30 bp; e.g., 3 bp, 4 bp, 5 bp, 6 bp, 7 bp, or 8 bp). For example, in any of the therapeutic circular DNA vectors generated using type IIs restriction enzymes, the 3′ end of the therapeutic sequence may be connected to the 5′ end of the therapeutic sequence by a non-bacterial sequence corresponding to sticky end or overhang of the type IIS restriction enzyme cut site (e.g., TTTT or AAAA).

In some embodiments, the therapeutic sequence includes a reporter sequence in addition to a therapeutic protein-encoding domain or a therapeutic non-protein encoding domain. Such reporter genes can be useful in verifying therapeutic gene sequence expression, for example, in specific cells and tissues. Reporter sequences that may be provided in a transgene include. without limitation. DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for B-galactosidase activity. Where the transgene is green fluorescent protein or luciferase, the vector carrying the signal may be measured visually by color or light production in a luminometer.

In some embodiments, the therapeutic sequence lacks a reporter sequence.

As part of the therapeutic sequence, therapeutic circular DNA vectors of the invention may include conventional control elements which modulate or improve transcription, translation, and/or expression in a target cell. Suitable control elements are described in International Publication No. WO 2021/055760, which is incorporated herein by reference in its entirety.

Pharmaceutical compositions may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and poloxamers, e.g., PLURONICS®.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂, Cal₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂) or potassium chloride (KCl), wherein further anions may be present. CaCl₂ can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂). The injection buffer may be hypertonic, isotonic, or hypotonic with reference to the specific reference medium, i.e., the buffer may have a higher, identical, or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

In some instances, the pharmaceutical composition is a viscous liquid.

In some instances, the pharmaceutical composition is an aerosol (e.g., upon release from the delivery port).

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from heobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

In some instances, the agent, or composition thereof, is administered locally through the delivery lumen.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

Synthetic circular DNA vectors according to the invention may be administered naked in a suitable buffer without being associated with any further vehicle, transfection, or complexation agent.

In certain embodiments of the invention, any of the nucleic acid vectors of the invention can be complexed with one or more cationic or polycationic compounds, e.g., cationic or polycationic polymers, cationic or polycationic peptides or proteins, e.g., protamine, cationic or polycationic polysaccharides, and/or cationic or polycationic lipids.

According to a particular embodiment, a nucleic acid vector of the invention may be complexed with lipids to form one or more liposomes, lipoplexes, or lipid nanoparticles. Therefore, in one embodiment, the pharmaceutical composition comprises liposomes, lipoplexes, and/or lipid nanoparticles including a nucleic acid vector.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for therapeutic circular DNA vectors. Cationic lipids, such as MAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

In a further embodiment of the invention, the nucleic acid vector is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the nucleic acid vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, a transfection agent, or a complexation agent for increasing the transfection efficiency and/or the expression of the sequence according to the invention.

In some instances, the nucleic acid vector is complexed with one or more polycations, preferably with protamine or oligofectamine. Further cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g. selected from a cationic polymer as mentioned above) and of one or more hydrophilic or hydrophobic blocks (e.g., polyethyleneglycole); etc.

According to a particular embodiment, the pharmaceutical composition includes the nucleic acid vector encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinking are typically selected from any suitable cationic or polycationic peptide, protein, or polymer suitable for this purpose, particularly any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the nucleic acid vector included as part of the pharmaceutical composition may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the nucleic acid vector may be formed by disulfide-crosslinked cationic (or polycationic) components. In particular embodiments, such cationic or polycationic peptides or proteins or polymers of the polymeric carrier, which comprise or are additionally modified to comprise at least one SH moiety, can be selected from proteins, peptides, and polymers as a complexation agent.

### EXAMPLES

### Example 1. Single-Balloon Device with a Peripheral Inflation Lumen

A single-balloon device features a central bypass tube and two peripheral tubes having an inflation lumen and a delivery lumen. FIG. 1 shows a slightly oblique side view of the device with the distal end at the top. The bypass lumen runs through the distal end. The distal end is at the tip of a chamfered tip (black), which sits distal to a balloon, shown in its deflated position in FIG. 1. The deflated balloon is a compliant, polyurethane sleeve bonded to the catheter at its proximal and distal ends. Near the middle of the axial length of the balloon, and sandwiched between the balloon and the catheter, is the distal end of an inflation lumen tube (i.e., the inflation port), providing access from the inflation lumen to the interior of the balloon (i.e., between the outer diameter of the catheter and the inner wall of the balloon).

Proximal to the balloon is a cylindrical mesh electrode wrapped around the circumference of the catheter. Near the proximal end of the mesh electrode is the distal end of a delivery lumen tube (i.e., the delivery port).

FIG. 2 shows a transverse cross-section of the catheter at a point proximal to the mesh electrode. The inflation lumen tube and the delivery lumen tube are bundled together with an elongate conductor, which connects to the mesh electrode at a lead. The inflation lumen tube, delivery lumen, and elongate conductor are bundled on one side of the periphery of a larger bypass lumen tube, and these components are held together by an outer shrink tube to form the catheter.

### Example 2. Single-Balloon Device with an Annular Inflation Lumen

A single-balloon device was designed featuring two central tubes (an inner tube to provide a bypass lumen and an outer tube to provide an inflation lumen) and a single peripheral tube to provide a delivery lumen. FIG. 3 shows a slightly oblique side view of the device with the distal end at the top. The bypass lumen runs through the distal end. The distal end is at the tip of a chamfered tip (black), which sits distal to a balloon, shown in its deflated position in FIG. 3. The deflated balloon is a compliant, polyurethane sleeve bonded to the catheter at the distal end of the balloon and to the outer tube of the catheter at the proximal end of the balloon. The inflation lumen is the annular space between the inner wall of the outer tube and the inner wall of the outer tube. The inflation port is the interface between the proximal end of the balloon and the distal end of the inflation lumen

Proximal to the balloon is a cylindrical electrode wrapped around the circumference of the catheter. At the proximal end of the electrode is the distal end of a delivery lumen tube, i.e., the delivery port.

FIG. 4 shows a transverse cross-section of the catheter at a point proximal to the electrode. The delivery lumen tube is bundled together with an elongate conductor, which connects to the electrode at a lead. The delivery lumen tube and elongate conductor are bundled on one side of the periphery of the outer tube, which serves as the inflation lumen tube, and these components are held together by an outer shrink tube. FIG. 4 shows the concentric arrangement of the two central tubes and their respective lumens, wherein the outer tube is the inflation lumen tube, the inner tube is the bypass lumen tube, the annular space between the inner wall of the outer tube and the inner wall of the outer tube is the inflation lumen, and lumen of the inner tube is the bypass lumen.

### Example 3. Use of a single-Balloon Device for Electrotransfer to Airway Epithelium

A single-balloon device is manufactured to have the features of the device of Example 1 or 2, such that the outer diameter of the catheter is about 10 mm, and the inner diameter of the bypass lumen is about 5 mm. In an operating room, a technician attaches the device to a waveform generator configured to transmit pulses of electrical energy through the elongate electrode to the electrode. The proximal end of the delivery lumen tube is connected to an inlet port, which is attached to a syringe containing a pharmaceutical composition containing naked circular DNA. The proximal end of the inflation lumen tube is connected to an inlet port, which is attached to a syringe containing an inflation medium (e.g., air).

A patient in need of the pharmaceutical composition is seated at a 90° upright position. A physician inserts a guidewire into the trachea of the patient and advances the device over the guidewire into the trachea. The distal end of the device is positioned where the trachea splits into the left and right primary bronchi, as shown in FIGS. 5A and 5B. The physician depresses the plunger on the inflation syringe to inflate the balloon such that the balloon seals the space between the outer wall of the device and the inner wall of the trachea, leaving the bypass lumen free to accommodate airflow sufficient for gas exchange (FIG. 5A). The pressure acting on the wall of the trachea from the balloon is sufficient to hold the weight of a predetermined volume of one dose of the agent in a pharmaceutical composition.

The physician depresses the plunger on the delivery syringe to administer the pharmaceutical composition to the treatment region. The pharmaceutical composition exits the delivery port and floods the space between the outer wall of the device and the inner wall of the trachea, from the balloon-trachea seal to the proximal end of the electrode such that the electrode is completely submerged in the pharmaceutical composition (FIG. 5B).

Within one minute after delivery of the pharmaceutical composition, a technician administers a series of voltage pulses using the waveform generator, each of which generates an electric field and a corresponding current at the electrode that is transmitted radially (and proximally and distally) relative to the catheter, thereby causing electrotransfer of the naked circular DNA in the pharmaceutical composition into the tracheal epithelial cells. After electrotransfer, the physician deflates the balloon and removes the device from the patient.

### Example 4. Dual-Balloon Device with a Single Inflation Lumen

A dual-balloon device was designed featuring two central tubes (an inner tube to provide a bypass lumen and a delivery lumen, and an outer tube to provide a single inflation lumen). FIG. 6 shows a side view of the device with the distal end at the top. The bypass lumen runs through the distal end. The distal end is at the tip of a chamfered tip (black), which sits distal to a distal balloon, shown in its deflated position in FIG. 6. Proximal to the balloon is a cylindrical mesh electrode wrapped around the circumference of the catheter. The delivery port passes through the outer tube of the catheter at a point near the terminal end of the mesh electrode. The mesh electrode covers the delivery port, but its permeability does not occlude the delivery port. Proximal to the mesh electrode is a proximal balloon shown in its deflated position.

Both balloons are compliant, polyurethane sleeves bonded to the outer tube at the ends of each balloon to form an airtight seal. The inflation lumen is the annular space between the inner wall of the outer tube and the inner wall of the outer tube. From proximal to distal directions, the inflation lumen runs from the proximal end of the catheter to the proximal end of the proximal balloon, continues through the proximal balloon, then continues from the distal end of the proximal balloon to the proximal end of the distal balloon. This single inflation lumen design is configured to inflate both balloons with a single inflation (e.g., near simultaneously).

FIG. 7 shows a transverse cross-section of the catheter at a point proximal to the proximal balloon. The outer tube houses the inner tube and an elongate conductor, which connects to the electrode at a lead. The inner tube is an integral construction of a central bypass lumen with a peripheral delivery lumen. The delivery lumen causes a protrusion from the outer wall of the inner tube, and the inner diameter of the outer tube is larger than the outer diameter of the inner tube by the amount of the protrusion. Such diameter difference creates a semi-annular space between the outer wall of the inner tube and the inner wall of the outer tube. This semi-annular space functions as the single inflation lumen and, additionally, houses the elongate conductor.

### Example 5. Dual-Balloon Device with Proximal and Distal Inflation Lumens

A dual-balloon device was designed featuring a single-tube catheter containing an elongate electrode and four lumens—a central bypass lumen and three peripheral lumens (i.e., a delivery lumen, a proximal inflation lumen, and a distal inflation lumen). FIG. 8 shows a slightly oblique side view of the device with the distal end at the top. The bypass lumen runs through the distal end. The distal end is at the tip of a chamfered tip (black), which sits distal to a distal balloon, shown in its deflated position. Proximal to the distal balloon is a cylindrical mesh electrode wrapped around the circumference of the catheter. The mesh electrode covers a delivery port, but its permeability does not occlude the delivery port. Proximal to the mesh electrode is a proximal balloon, shown in its deflated position. Both balloons are compliant, polyurethane sleeves bonded to the catheter at the ends of each balloon to form an airtight seal.

FIG. 9A shows a transverse cross-section of the device at the proximal balloon. The proximal balloon is shown as a white circle surrounding the dark central tube of the catheter. The central tube contains a central lumen that serves as the bypass lumen. Integrated at the bottom of the central tube by coextrusion is an elongate conductor, which connects to the electrode at the lead. Integrated at the top of the central tube is a delivery lumen. Integrated at either side of the central tube is a proximal inflation lumen and a distal inflation lumen, respectively.

FIG. 9B shows a transverse cross-section of the catheter at the electrode (the electrode is not shown). What was the delivery lumen in FIG. 9A has become the delivery port in FIG. 9B—the lumen has opened to the periphery. Similarly, what was the elongate conductor in FIG. 9A has become the lead slot in FIG. 9B. The electrode attaches to the elongate conductor through a lead (not shown) positioned in the lead slot.

FIG. 9C shows a transverse cross-section of the catheter at the distal balloon (balloon not shown). What were the delivery port, proximal inflation lumen, and lead slot in FIG. 9B are filled in with the catheter material at this distal position shown in FIG. 9C. The only remaining lumens are the bypass lumen, which runs through the distal end of the device, and the distal inflation lumen, which terminates at a distal inflation port at the distal balloon.

### Example 6. Use of a Dual-Balloon Device for Electrotransfer to Airway Epithelium

A dual-balloon device is manufactured to have the features of the device of Example 4 or 5, such that the outer diameter of the catheter is about 10 mm and the inner diameter of the bypass lumen is about 7 mm. In an operating room, a technician attaches the device to a waveform generator configured to transmit pulses of electrical energy through the elongate electrode to the electrode. The proximal end of the delivery lumen tube is connected to an inlet port, which is attached to a syringe containing a pharmaceutical composition containing naked circular DNA. The proximal end of the inflation lumen is connected to an inlet port, which is attached to a syringe containing air as an inflation medium.

A patient in need of the pharmaceutical composition is in a supine position for the procedure. A physician inserts a guidewire into the trachea of the patient and advances the device over the guidewire into the trachea. The distal end of the device is positioned where the trachea splits into the left and right primary bronchi, as shown in FIGS. 10A and 10B. The physician depresses the plunger on the inflation syringe to inflate the balloons such that each balloon seals the space between the outer wall of the device and the inner wall of the trachea, leaving the bypass lumen free to accommodate airflow sufficient for gas exchange (FIG. 10A). The space between the two balloons define the treatment region.

The physician depresses the plunger on the delivery syringe to administer the pharmaceutical composition to the treatment region. The pharmaceutical composition exits the delivery port and floods the space between the outer wall of the device and the inner wall of the trachea, from the distal balloon-trachea seal to the proximal balloon-trachea seal, such that the electrode is completely submerged in the pharmaceutical composition (FIG. 10B).

Within one minute after delivery of the pharmaceutical composition, a technician administers a series of voltage pulses using the waveform generator, each of which generates current at the electrode which is transmitted radially (and proximally and distally) relative to the catheter, thereby causing electrotransfer of the naked circular DNA in the pharmaceutical composition into the tracheal epithelial cells in the treatment region. After electrotransfer, the physician deflates the balloons and removes the device from the patient.

### Example 7. In Vivo Delivery of Pulsed Electric Fields to Trachea and Upper Bronchi

The device shown in FIGS. 12A and 12B (single balloon distal to the electrode, as illustrated schematically in FIG. 5A) was used to deliver electrical pulses to a rabbit airway. The device contained a catheter of approximately 7 F inner diameter (bypass lumen) to allow breathing and 9 F outer diameter. The proximal end was fitted with a standard 15 mm respirator fitting to facilitate respiration. The balloon was bonded to the catheter shaft at the distal portion of the device to prevent test solution from leaking distally into the airway. The catheter includes an inflation lumen running along the shaft to provide air to the balloon. A separate lumen runs along the shaft of the catheter to deliver the test solution. An electrode wire is wrapped around the circumference of the shaft of the catheter beginning proximal to the balloon and extending 20 mm, defining the target area in which the electrical current is applied. Because the therapeutic solution conducts the electrical signal, the electrode does not need to extend the entire length of the treatment zone.

The following procedure was conducted for each of the five animals (male New Zealand white rabbits) of this study.

Animals were fasted overnight prior to the procedure. Hair was removed from the animal's neck and upper torso. A three-lead EKG was affixed to the animal to monitor heart rhythms. The animal was placed in the prone position and limbs secured to the table. A grounding pad was attached to the animal's neck or torso. The animal's teeth were covered with gauze or wax to prevent damage to the device. The animal was anesthetized using a pre-medication of Xylazine (0.5 mg/kg Intra-muscular (IM) administration) and anesthetize with ketamine (22 mg/kg IM).

First, the device was attached to a pulse generator (BTX). A ventilator was set for use at 30-60 breaths per minute with 100% oxygen, between 6-10 ml/kg tidal volume, and a 15 mm female fitting was attached to the ventilator.

An R4 sized V-gel endotracheal tube was inserted into the airway using standard techniques. A straight guidewire (0.035″ or 0.018″) was advanced past the end of the V-gel endotracheal tube to sit a few centimeters distal to the larynx. A surgical lubricant was applied to the distal tip and balloon. The catheter was advanced over the guidewire to into the airway. When the tip of the catheter is above the larynx, 1 cm markings on the catheter were used to measure the depth of the larynx from the tip of the animal's nose/mouth. Once the proximal balloon is positioned distal to the larynx, the guidewire was removed. The catheter was advanced 4 cm using the markings until the proximal balloon was about 4-5 cm distal to the larynx.

Next, the ventilator was attached to the proximal end of the catheter. The distal balloon was inflated to between 10 and 14 psi using an inflation syringe and gauge. The stopcock was twisted after complete inflation to maintain pressure in the distal balloon, and respiration through the main lumen was confirmed.

As a paralytic, vecuronium was administered intravenously at 0.5 mg/kg. The animal's SpO₂ was monitored.

The animal's head was held up to prevent liquid from flowing out of the airway. and approximately 600 microliters of the test fluid was injected into the trachea over the course of a few seconds.

Pulsed electric fields were applied (4 pulses total at 300 V each, 5 ms duration of each pulse). Twitching of the animal body confirmed that current was flowing from the electrode through the test solution into the animal's tissue.

After pulsed electric field transfer, test solution was aspirated back through the delivery lumen until air bubbles were observed in the syringe. The balloon was deflated over the course of 10 seconds, the device was withdrawn. The animal was allowed to recover until it breathed naturally before the endotracheal tube was removed.

This procedure was conducted on five animals. Successful ventilation and pulsed electric field-mediated current delivery was observed in three of the five animals; in the other two animals, the device was inserted incorrectly (into the esophagus), and the procedure was aborted. These observations indicated that in vivo delivery of pulsed electric field delivers therapeutic agents into the mammalian airway using a device of the invention.

### ORDERED EMBODIMENTS

The following sections describe various embodiments of the invention.

1. A device comprising:
(a) a catheter comprising:
   (i) a proximal end and a distal end;
   (ii) a delivery lumen that runs from the proximal end of the catheter to a delivery port proximal to the distal end of the catheter;
   (iv) an elongate conductor that runs from the proximal end of the catheter to a lead proximal to the distal end of the catheter; and
   (v) an inflation lumen that runs from the proximal end of the catheter to an inflation port proximal to the distal end of the catheter;
(b) a distal balloon disposed about the catheter over the distal inflation port and distal to the delivery port, wherein distal airflow through the inflation lumen inflates the distal balloon; and
(c) an electrode disposed about the catheter proximal to the balloon, wherein the electrode is connected to the lead.

2. The device of embodiment 1, wherein the catheter further comprises:
(iv) a bypass lumen that runs from the proximal end of the catheter through the distal end of the catheter.

3. The device of embodiment 1 or 2, wherein the electrode wraps around the circumference of the catheter.

4. The device of any one of embodiments 1-3, whereupon application of a voltage to the electrode generates an electric field and a corresponding current that flows radially relative to the catheter.

5. The device of any one of embodiments 1-4, wherein the electrode is a mesh, a spiral, or a combination thereof.

6. The device of any one of embodiments 1-5, wherein the axial length of the electrode is from two-fold to 20-fold the outer diameter of the catheter.

7. The device of embodiment 6, wherein the axial length of the electrode is about five-fold the outer diameter of the catheter.

8. The device of any one of embodiments 1-7, wherein the electrode comprises multiple electrically independent segments.

9. The device of any one of embodiments 1-8, wherein the distal balloon comprises an elastic material.

10. The device of embodiment 9, wherein the elastic material comprises polyurethane.

11. The device of any one of embodiments 1-10, wherein the axial length of the distal balloon is from one-fold to ten-fold the outer diameter of the catheter.

12. The device of any one of embodiments 1-11, wherein the distal balloon is radially inflatable to a diameter from 20% to 500% greater than the outer diameter of the distal balloon in its relaxed shape.

13. The device of any one of embodiments 1-12, wherein the distal balloon is a thin-walled polyurethane tube running from a proximal step down in the catheter to a distal step up in the catheter, wherein the outer diameter of the deflated distal balloon is substantially flush with the outer diameter of the catheter.

14. The device of any one of embodiments 1-13, wherein the distal balloon comprises a lubricious coating.

15. The device of any one of embodiments 1-14, wherein the distal balloon comprises a pressure sensor.

16. The device of any one of embodiments 1-15, wherein the catheter comprises an inner tube and an outer tube, wherein one or more inner lumens are disposed within the inner tube and one or more outer lumens are disposed between the outer wall of the inner tube and the inner wall of the outer tube.

17. The device of embodiment 16, wherein the elongate conductor is disposed within one of the one or more the outer lumens.

18. The device of embodiment 16 or 17, wherein the one or more outer lumens comprises the inflation lumen.

19. The device of any one of embodiments 16-18, wherein the one or more inner lumens comprises the delivery lumen.

20. The device of any one of embodiments 16-19, wherein the one or more inner lumens comprises the bypass lumen.

21. The device of any one of embodiments 1-20, wherein the elongate conductor is disposed within an insulating sleeve.

22. The device of embodiment 21, wherein the insulating sleeve comprises polyimide.

23. The device of any one of embodiments 1-22, wherein the distal end of the catheter comprises an atraumatic tip.

24. The device of any one of embodiments 1-23, wherein the proximal end of the catheter is connected to one or more inlet ports.

25. A device comprising:
(a) a catheter comprising:
   (i) a proximal end and a distal end;
   (ii) a delivery lumen that runs from the proximal end of the catheter to a delivery port proximal to the distal end of the catheter;
   (iv) an elongate conductor that runs from the proximal end of the catheter to a lead proximal to the distal end of the catheter; and
   (v) one or more inflation lumens that run from the proximal end of the catheter to a proximal inflation port proximal to the delivery port and a distal inflation port distal to the delivery port, wherein the distal inflation port is proximal to the distal end of the catheter;
(b) a proximal balloon disposed about the catheter over the proximal inflation port;
(c) a distal balloon disposed about the catheter over the distal inflation port, wherein distal airflow through the inflation lumen inflates the distal balloon; and
(d) an electrode disposed about the catheter proximal to the balloon, wherein the electrode is connected to the lead.

26. The device of embodiment 25, wherein the catheter further comprises:
(iv) a bypass lumen that runs from the proximal end of the catheter through the distal end of the catheter.

27. The device of embodiment 25 or 26, wherein the one or more inflation lumens comprises a proximal inflation lumen that runs to the proximal inflation port and a distal inflation lumen that runs to the distal inflation port.

28. The device of any one of embodiments 25-27, wherein the proximal balloon is independently inflatable relative to the distal balloon.

29. The device of embodiment 28, wherein the catheter comprises a single inflation lumen that runs to both the proximal inflation port and the distal inflation port.

30. The device of any one of embodiments 25-29, wherein the electrode wraps around the circumference of the catheter.

31. The device of any one of embodiments 25-30, whereupon application of a voltage to the electrode generates a current that flows radially relative to the catheter.

32. The device of any one of embodiments 25-31, wherein the electrode is a mesh, a spiral, or a combination thereof.

33. The device of any one of embodiments 25-32, wherein the axial length of the electrode is from two-fold to 20-fold the outer diameter of the catheter.

34. The device of embodiment 33, wherein the axial length of the electrode is about five-fold the outer diameter of the catheter.

35. The device of any one of embodiments 25-34, wherein the electrode comprises multiple electrically independent segments.

36. The device of any one of embodiments 25-35, wherein the distal balloon and/or the proximal balloon comprise an elastic material.

37. The device of embodiment 36, wherein the elastic material comprises polyurethane.

38. The device of any one of embodiments 25-37, wherein the axial length of each of the distal balloon and/or the proximal balloon is from one-fold to ten-fold the outer diameter of the catheter.

39. The device of any one of embodiments 25-38, wherein the distal balloon and/or the proximal balloon is radially inflatable to a diameter from 20% to 500% greater than the outer diameter of distal balloon and/or proximal balloon in its relaxed shape.

40. The device of any one of embodiments 25-39, wherein the distal balloon and/or the proximal balloon are thin-walled polyurethane tubes running from a proximal step down in the catheter to a distal step up in the catheter, wherein the outer diameter of the deflated balloon is flush with the outer diameter of the catheter.

41. The device of any one of embodiments 25-40, wherein the distal balloon and/or the proximal balloon comprises a lubricious coating.

42. The device of any one of embodiments 25-41, wherein the distal balloon and/or the proximal balloon comprises a pressure sensor.

43. The device of any one of embodiments 25-42, wherein the catheter comprises an inner tube and an outer tube, wherein one or more inner lumens are disposed within the inner tube and one or more outer lumens are disposed between the outer wall of the inner tube and the inner wall of the outer tube.

44. The device of embodiment 43, wherein the elongate conductor is disposed within one of the one or more the outer lumens.

45. The device of embodiment 43 or 44, wherein the one or more outer lumens comprise the distal inflation lumen and/or the proximal inflation lumen.

46. The device of any one of embodiments 43-45, wherein the one or more inner lumens comprise the delivery lumen.

47. The device of any one of embodiments 43-46, wherein the one or more inner lumens comprise the bypass lumen.

48. The device of any one of embodiments 25-47, wherein the elongate conductor is disposed within an insulating sleeve.

49. The device of embodiment 48, wherein the insulating sleeve comprises polyimide.

50. The device of any one of embodiments 25-49, wherein the distal end of the catheter comprises an atraumatic tip.

51. The device of any one of embodiments 25-50, wherein the proximal end of the catheter is connected to one or more inlet ports.

52. A system comprising the device of any one of embodiments 1-51 and a waveform generator connected to the elongate conductor.

53. A method of exposing a target area in a hollow tissue to electrical energy, the method comprising:
(a) providing a device comprising:
   (i) a catheter comprising:
      a proximal end and a distal end,
         a delivery lumen that runs from the proximal end of the catheter to a delivery port proximal to the distal end of the catheter,
         an elongate conductor that runs from the proximal end of the catheter to a lead proximal to the distal end of the catheter, and
         an inflation lumen that run from the proximal end of the catheter to an inflation port distal to the delivery port, wherein the distal inflation port is proximal to the distal end of the catheter;
   (ii) a balloon disposed about the catheter over the inflation port; and
   (iii) an electrode disposed about the catheter proximal to the balloon, wherein the electrode is connected to the lead;
(b) positioning the electrode within the hollow tissue, wherein a region of the hollow tissue radially adjacent to the electrode comprises all or a portion of the target area;
(c) flowing an inflation medium distally through the inflation lumen to inflate the balloon such that the balloon contacts the hollow tissue;
(d) introducing an agent through the delivery lumen and the delivery port such that the agent contacts the target area; and
(e) while the balloon is inflated, transmitting electrical energy through the electrode, thereby exposing the target area to the electrical energy.

54. The method of embodiment 53, wherein the electrical energy is transmitted at conditions suitable for electrotransfer of the agent into a target cell in the target area.

55. A method of delivering an agent to a target cell in a hollow tissue, the method comprising:
(a) providing a device comprising:
   (i) a catheter comprising:
      a proximal end and a distal end,
         a delivery lumen that runs from the proximal end of the catheter to a delivery port proximal to the distal end of the catheter,
         an elongate conductor that runs from the proximal end of the catheter to a lead proximal to the distal end of the catheter, and
         an inflation lumen that run from the proximal end of the catheter to an inflation port distal to the delivery port, wherein the distal inflation port is proximal to the distal end of the catheter;
   (ii) a balloon disposed about the catheter over the inflation port; and
   (iii) an electrode disposed about the catheter proximal to the balloon, wherein the electrode is connected to the lead;
(b) positioning the electrode within the hollow tissue, wherein a region of the hollow tissue radially adjacent to the electrode comprises the target cell;
(c) flowing an inflation medium distally through the inflation lumen to inflate the balloon such that the balloon contacts the hollow tissue;
(d) introducing the agent through the delivery lumen and the delivery port such that the agent contacts the region of the hollow tissue radially adjacent to the electrode; and
(e) while the balloon is inflated, transmitting electrical energy through the electrode at conditions suitable for electrotransfer of the agent into the target cell.

56. The method of any one of embodiments 53-55, wherein the agent is a therapeutic agent.

57. The method of any one of embodiments 53-56, wherein the agent is a nucleic acid vector.

58. The method of embodiment 57, wherein the nucleic acid vector is expressed in the hollow tissue.

59. A method of expressing a nucleic acid vector in a target cell in a hollow tissue, the method comprising:
(a) providing a device comprising:
   (i) a catheter comprising:
      a proximal end and a distal end,
         a delivery lumen that runs from the proximal end of the catheter to a delivery port proximal to the distal end of the catheter,
         an elongate conductor that runs from the proximal end of the catheter to a lead proximal to the distal end of the catheter, and
         an inflation lumens that run from the proximal end of the catheter to an inflation port distal to the delivery port, wherein the distal inflation port is proximal to the distal end of the catheter;
   (ii) a balloon disposed about the catheter over the inflation port; and
   (iii) an electrode disposed about the catheter proximal to the balloon, wherein the electrode is connected to the lead;
(b) positioning the electrode within the hollow tissue, wherein a region of the hollow tissue radially adjacent to the electrode comprises the target cell;
(c) flowing an inflation medium distally through the inflation lumen to inflate the balloon such that the balloon contacts the hollow tissue;
(d) introducing the nucleic acid vector through the delivery lumen and the delivery port such that the nucleic acid vector contacts the region of the hollow tissue radially adjacent to the electrode;
(e) while the balloon is inflated, transmitting electrical energy through the electrode at conditions suitable for electrotransfer of the nucleic acid vector into the target cell; and
(f) detecting expression of the nucleic acid vector by the target cell.

60. A method of treating a disease or disorder in an individual, the method comprising:
(a) providing a device comprising:
   (i) a catheter comprising:
      a proximal end and a distal end,
         a delivery lumen that runs from the proximal end of the catheter to a delivery port proximal to the distal end of the catheter,
         an elongate conductor that runs from the proximal end of the catheter to a lead proximal to the distal end of the catheter, and
         an inflation lumens that run from the proximal end of the catheter to an inflation port distal to the delivery port, wherein the distal inflation port is proximal to the distal end of the catheter;
   (ii) a balloon disposed about the catheter over the inflation port; and
   (iii) an electrode disposed about the catheter proximal to the balloon, wherein the electrode is connected to the lead;
(b) positioning the electrode within a hollow tissue in the individual, wherein a region of the hollow tissue radially adjacent to the electrode comprises a target cell;
(c) flowing an inflation medium distally through the inflation lumen to inflate the balloon such that the balloon contacts the hollow tissue;
(d) introducing an effective amount of therapeutic agent through the delivery lumen and the delivery port such that the therapeutic agent contacts the region of the hollow tissue radially adjacent to the electrode; and
(e) while the balloon is inflated, transmitting electrical energy through the electrode at conditions suitable for electrotransfer of an effective amount of the therapeutic agent into the target cell, thereby treating the disease or disorder.

61. The method of embodiment 60, wherein the therapeutic agent is a nucleic acid vector.

62. The method of any one of embodiments 57-61, wherein the nucleic acid vector comprises a therapeutic protein-encoding sequence.

63. The method of embodiment 62, wherein the therapeutic protein is a therapeutic replacement protein.

64. The method of any one of embodiments 60-63, wherein the individual is monitored for progression of the disease or disorder after the treatment.

65. A method of exposing a target area in a hollow tissue to electrical energy, the method comprising:
(a) providing a device comprising:
   (i) a catheter comprising:
      a proximal end and a distal end;
         a delivery lumen that runs from the proximal end of the catheter to a delivery port proximal to the distal end of the catheter;
         an elongate conductor that runs from the proximal end of the catheter to a lead proximal to the distal end of the catheter; and
         one or more inflation lumens that run from the proximal end of the catheter to a proximal inflation port proximal to the delivery port and a distal inflation port distal to the delivery port, wherein the distal inflation port is proximal to the distal end of the catheter;
   (ii) a proximal balloon disposed about the catheter over the proximal inflation port;
   (iii) a distal balloon disposed about the catheter over the distal inflation port; and
   (iv) an electrode disposed about the catheter proximal to the balloon, wherein the electrode is connected to the lead;
(b) positioning the electrode within the hollow tissue, wherein the region of the hollow tissue radially adjacent to the electrode comprises all or a portion of the target area;
(c) flowing an inflation medium distally through the one or more inflation lumens to inflate the proximal balloon and the distal balloon such that each of the proximal balloon and distal balloon contacts the hollow tissue;
(d) introducing an agent through the delivery lumen and the delivery port such that the agent contacts the target area; and
(e) while the proximal and distal balloons are inflated, transmitting electrical energy through the electrode, thereby exposing the target area to the electrical energy.

66. The method of embodiment 65, wherein the electrical energy is transmitted at conditions suitable for electrotransfer of the agent into a target cell in the target area.

67. A method of delivering an agent to a target cell in a hollow tissue, the method comprising:
(a) providing a device comprising:
   (i) a catheter comprising:
      a proximal end and a distal end;
         a delivery lumen that runs from the proximal end of the catheter to a delivery port proximal to the distal end of the catheter;
         an elongate conductor that runs from the proximal end of the catheter to a lead proximal to the distal end of the catheter; and
         one or more inflation lumens that run from the proximal end of the catheter to a proximal inflation port proximal to the delivery port and a distal inflation port distal to the delivery port, wherein the distal inflation port is proximal to the distal end of the catheter;
   (ii) a proximal balloon disposed about the catheter over the proximal inflation port;
   (iii) a distal balloon disposed about the catheter over the distal inflation port; and
   (iv) an electrode disposed about the catheter proximal to the balloon, wherein the electrode is connected to the lead;
(b) positioning the electrode within the hollow tissue, wherein the region of the hollow tissue radially adjacent to the electrode comprises the target cell;
(c) flowing an inflation medium distally through the one or more inflation lumens to inflate the proximal balloon and the distal balloon such that each of the proximal balloon and distal balloon contacts the hollow tissue;
(d) introducing the agent through the delivery lumen and the delivery port such that the agent contacts the region of the hollow tissue radially adjacent to the electrode; and
(e) while the proximal and distal balloons are inflated, transmitting electrical energy through the electrode at conditions suitable for electrotransfer of the agent into the target cell.

68. The method of any one of embodiments 65-67, wherein the agent is a therapeutic agent.

69. The method of any one of embodiments 65-68, wherein the agent is a nucleic acid vector.

70. The method of embodiment 69, wherein the nucleic acid vector is expressed in the hollow tissue.

71. A method of expressing a nucleic acid vector in a target cell in a hollow tissue, the method comprising:
(a) providing a device comprising:
   (i) a catheter comprising:
      a proximal end and a distal end;
         a delivery lumen that runs from the proximal end of the catheter to a delivery port proximal to the distal end of the catheter;
         an elongate conductor that runs from the proximal end of the catheter to a lead proximal to the distal end of the catheter; and
         one or more inflation lumens that run from the proximal end of the catheter to a proximal inflation port proximal to the delivery port and a distal inflation port distal to the delivery port, wherein the distal inflation port is proximal to the distal end of the catheter;
   (ii) a proximal balloon disposed about the catheter over the proximal inflation port;
   (iii) a distal balloon disposed about the catheter over the distal inflation port; and
   (iv) an electrode disposed about the catheter proximal to the balloon, wherein the electrode is connected to the lead;
(b) positioning the electrode within the hollow tissue, wherein the region of the hollow tissue radially adjacent to the electrode comprises the target cell;
(c) flowing an inflation medium distally through the one or more inflation lumens to inflate the proximal balloon and the distal balloon such that each of the proximal balloon and distal balloon contacts the hollow tissue;
(d) introducing the nucleic acid vector through the delivery lumen and the delivery port such that the nucleic acid vector contacts the region of the hollow tissue radially adjacent to the electrode;
(e) while the proximal and distal balloons are inflated, transmitting electrical energy through the electrode at conditions suitable for electrotransfer of the nucleic acid vector into the target cell; and
(f) detecting expression of the nucleic acid vector by the target cell.

72. A method of treating a disease or disorder in an individual, the method comprising:
(a) providing a device comprising:
   (i) a catheter, wherein the catheter comprises:
      a proximal end and a distal end;
         a delivery lumen that runs from the proximal end of the catheter to a delivery port proximal to the distal end of the catheter;
         an elongate conductor that runs from the proximal end of the catheter to a lead proximal to the distal end of the catheter; and
         one or more inflation lumens that run from the proximal end of the catheter to a proximal inflation port proximal to the delivery port and a distal inflation port distal to the delivery port, wherein the distal inflation port is proximal to the distal end of the catheter;
   (ii) a proximal balloon disposed about the catheter over the proximal inflation port;
   (iii) a distal balloon disposed about the catheter over the distal inflation port; and
   (iv) an electrode disposed about the catheter proximal to the balloon, wherein the electrode is connected to the lead;
(b) positioning the electrode within a hollow tissue in the individual, wherein the region of the hollow tissue radially adjacent to the electrode comprises a target cell;
(c) flowing an inflation medium distally through the one or more inflation lumens to inflate the proximal balloon and the distal balloon such that each of the proximal balloon and distal balloon contacts the hollow tissue;
(d) introducing an effective amount of a therapeutic agent through the delivery lumen and the delivery port such that the therapeutic agent contacts the region of the hollow tissue radially adjacent to the electrode; and
(e) while the proximal and distal balloons are inflated, transmitting electrical energy through the electrode at conditions suitable for electrotransfer of an effective amount of the therapeutic agent into the target cell, thereby treating the disease or disorder.

73. The method of embodiment 72, wherein the therapeutic agent is a nucleic acid vector.

74. The method of any one of embodiments 69-73, wherein the nucleic acid vector comprises a therapeutic protein-encoding sequence.

75. The method of embodiment 74, wherein the therapeutic protein is a therapeutic replacement protein.

76. The method of any one of embodiments 72-75, wherein the individual is monitored for progression of the disease or disorder after the treatment.

77. The method of embodiment 72, wherein the one or more inflation lumens comprises a proximal inflation lumen that runs to the proximal inflation port and a distal inflation lumen that runs to the distal inflation port.

78. The method of any one of embodiments 65-77, wherein step (c) comprises inflating the proximal balloon and the distal balloon simultaneously.

79. The method of any one of embodiments 65-78, wherein step (c) comprises inflating the proximal balloon independently from the distal balloon.

80. The method of embodiment 79, wherein step (c) comprises inflating the proximal balloon after inflating the distal balloon.

81. The method of any one of embodiments 65-80, wherein the catheter comprises a single inflation lumen that runs to both the proximal inflation port and the distal inflation port.

82. The method of any one of embodiments 65-81, wherein the hollow tissue is a tubular tissue.

83. The method of embodiment 82, wherein the catheter further comprises a bypass lumen that runs from the proximal end of the catheter through the distal end of the catheter, wherein the method is performed without occluding flow through the tubular tissue.

84. The method of embodiment 82 or 83, wherein the hollow tissue is an airway.

85. The method of embodiment 84, wherein the airway is a trachea, a bronchus, or a bronchiole.

86. The method of any one of embodiments 55-64 or 67-85, wherein the target cell is an airway epithelial cell.

87. The method of any one of embodiments 53-86, wherein the agent is formulated as a liquid.

88. The method of embodiment 87, wherein the liquid is a viscous liquid.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A device comprising:
(a) a catheter comprising:
(i) a proximal end and a distal end;
(ii) a delivery lumen that runs from the proximal end of the catheter to a delivery port proximal to the distal end of the catheter;
(iv) an elongate conductor that runs from the proximal end of the catheter to a lead proximal to the distal end of the catheter; and
(v) an inflation lumen that runs from the proximal end of the catheter to an inflation port proximal to the distal end of the catheter;
(b) a distal balloon disposed about the catheter over the distal inflation port and distal to the delivery port, wherein distal airflow through the inflation lumen inflates the distal balloon; and
(c) an electrode disposed about the catheter proximal to the balloon, wherein the electrode is connected to the lead.

2. The device of claim 1, wherein the catheter further comprises:
(iv) a bypass lumen that runs from the proximal end of the catheter through the distal end of the catheter.

3. The device of claim 1, wherein the electrode wraps around the circumference of the catheter.

4. The device of claim 1, whereupon application of a voltage to the electrode generates an electric field and a corresponding current that flows radially relative to the catheter.

5. The device of claim 1, wherein the electrode is a mesh, a spiral, or a combination thereof.

6. The device of claim 1, wherein the axial length of the electrode is from two-fold to 20-fold the outer diameter of the catheter.

7. The device of claim 6, wherein the axial length of the electrode is about five-fold the outer diameter of the catheter.

8. The device of claim 1, wherein the electrode comprises multiple electrically independent segments.

9. The device of claim 1, wherein the distal balloon comprises an elastic material.

10. The device of claim 9, wherein the elastic material comprises polyurethane.

11. The device of claim 1, wherein the axial length of the distal balloon is from one-fold to ten-fold the outer diameter of the catheter.

12. The device of claim 1, wherein the distal balloon is radially inflatable to a diameter from 20% to 500% greater than the outer diameter of the distal balloon in its relaxed shape.

13. The device of claim 1, wherein the distal balloon is a thin-walled polyurethane tube running from a proximal step down in the catheter to a distal step up in the catheter, wherein the outer diameter of the deflated distal balloon is substantially flush with the outer diameter of the catheter.

14. The device of claim 1, wherein the distal balloon comprises a lubricious coating.

15. The device of claim 1, wherein the distal balloon comprises a pressure sensor.

16. The device of claim 1, wherein the catheter comprises an inner tube and an outer tube, wherein one or more inner lumens are disposed within the inner tube and one or more outer lumens are disposed between the outer wall of the inner tube and the inner wall of the outer tube.

17. The device of claim 16, wherein the elongate conductor is disposed within one of the one or more the outer lumens.

18. The device of claim 16, wherein the one or more outer lumens comprises the inflation lumen.

19. The device of claim 16, wherein the one or more inner lumens comprises the delivery lumen.

20. The device of claim 16, wherein the one or more inner lumens comprises the bypass lumen.

21. The device of claim 1, wherein the elongate conductor is disposed within an insulating sleeve.

22. The device of claim 21, wherein the insulating sleeve comprises polyimide.

23. The device of claim 1, wherein the distal end of the catheter comprises an atraumatic tip.

24. The device of claim 1, wherein the proximal end of the catheter is connected to one or more inlet ports.

25. A device comprising:
(a) a catheter comprising:
(i) a proximal end and a distal end;
(ii) a delivery lumen that runs from the proximal end of the catheter to a delivery port proximal to the distal end of the catheter;
(iv) an elongate conductor that runs from the proximal end of the catheter to a lead proximal to the distal end of the catheter; and
(v) one or more inflation lumens that run from the proximal end of the catheter to a proximal inflation port proximal to the delivery port and a distal inflation port distal to the delivery port, wherein the distal inflation port is proximal to the distal end of the catheter;
(b) a proximal balloon disposed about the catheter over the proximal inflation port;
(c) a distal balloon disposed about the catheter over the distal inflation port, wherein distal airflow through the inflation lumen inflates the distal balloon; and
(d) an electrode disposed about the catheter proximal to the balloon, wherein the electrode is connected to the lead.

26. The device of claim 25, wherein the catheter further comprises:
(iv) a bypass lumen that runs from the proximal end of the catheter through the distal end of the catheter.

27. The device of claim 25, wherein the one or more inflation lumens comprises a proximal inflation lumen that runs to the proximal inflation port and a distal inflation lumen that runs to the distal inflation port.

28. The device of claim 25, wherein the proximal balloon is independently inflatable relative to the distal balloon.

29. The device of claim 28, wherein the catheter comprises a single inflation lumen that runs to both the proximal inflation port and the distal inflation port.

30. The device of claim 25, wherein the electrode wraps around the circumference of the catheter.

31. The device of claim 25, whereupon application of a voltage to the electrode generates a current that flows radially relative to the catheter.

32. The device of claim 25, wherein the electrode is a mesh, a spiral, or a combination thereof.

33. The device of claim 25, wherein the axial length of the electrode is from two-fold to 20-fold the outer diameter of the catheter.

34. The device of claim 33, wherein the axial length of the electrode is about five-fold the outer diameter of the catheter.

35. The device of claim 25, wherein the electrode comprises multiple electrically independent segments.

36. The device of claim 25, wherein the distal balloon and/or the proximal balloon comprise an elastic material.

37. The device of claim 36, wherein the elastic material comprises polyurethane.

38. The device of claim 25, wherein the axial length of each of the distal balloon and/or the proximal balloon is from one-fold to ten-fold the outer diameter of the catheter.

39. The device of claim 25, wherein the distal balloon and/or the proximal balloon is radially inflatable to a diameter from 20% to 500% greater than the outer diameter of distal balloon and/or proximal balloon in its relaxed shape.

40. The device of claim 25, wherein the distal balloon and/or the proximal balloon are thin-walled polyurethane tubes running from a proximal step down in the catheter to a distal step up in the catheter, wherein the outer diameter of the deflated balloon is flush with the outer diameter of the catheter.

41. The device of claim 25, wherein the distal balloon and/or the proximal balloon comprises a lubricious coating.

42. The device of claim 25, wherein the distal balloon and/or the proximal balloon comprises a pressure sensor.

43. The device of claim 25, wherein the catheter comprises an inner tube and an outer tube, wherein one or more inner lumens are disposed within the inner tube and one or more outer lumens are disposed between the outer wall of the inner tube and the inner wall of the outer tube.

44. The device of claim 43, wherein the elongate conductor is disposed within one of the one or more the outer lumens.

45. The device of claim 43, wherein the one or more outer lumens comprise the distal inflation lumen and/or the proximal inflation lumen.

46. The device of claim 43, wherein the one or more inner lumens comprise the delivery lumen.

47. The device of claim 43, wherein the one or more inner lumens comprise the bypass lumen.

48. The device of claim 25, wherein the elongate conductor is disposed within an insulating sleeve.

49. The device of claim 48, wherein the insulating sleeve comprises polyimide.

50. The device of claim 25, wherein the distal end of the catheter comprises an atraumatic tip.

51. The device of claim 25, wherein the proximal end of the catheter is connected to one or more inlet ports.

52. A system comprising the device of claim 1 and a waveform generator connected to the elongate conductor.

53. A method of exposing a target area in a hollow tissue to electrical energy, the method comprising:
(a) providing a device comprising:
(i) a catheter comprising:
a proximal end and a distal end,
a delivery lumen that runs from the proximal end of the catheter to a delivery port proximal to the distal end of the catheter,
an elongate conductor that runs from the proximal end of the catheter to a lead proximal to the distal end of the catheter, and
an inflation lumen that run from the proximal end of the catheter to an inflation port distal to the delivery port, wherein the distal inflation port is proximal to the distal end of the catheter;
(ii) a balloon disposed about the catheter over the inflation port; and
(iii) an electrode disposed about the catheter proximal to the balloon, wherein the electrode is connected to the lead;
(b) positioning the electrode within the hollow tissue, wherein a region of the hollow tissue radially adjacent to the electrode comprises all or a portion of the target area;
(c) flowing an inflation medium distally through the inflation lumen to inflate the balloon such that the balloon contacts the hollow tissue;
(d) introducing an agent through the delivery lumen and the delivery port such that the agent contacts the target area; and
(e) while the balloon is inflated, transmitting electrical energy through the electrode, thereby exposing the target area to the electrical energy.

54. The method of claim 53, wherein the electrical energy is transmitted at conditions suitable for electrotransfer of the agent into a target cell in the target area.

55. A method of delivering an agent to a target cell in a hollow tissue, the method comprising:
(a) providing a device comprising:
(i) a catheter comprising:
a proximal end and a distal end,
a delivery lumen that runs from the proximal end of the catheter to a delivery port proximal to the distal end of the catheter,
an elongate conductor that runs from the proximal end of the catheter to a lead proximal to the distal end of the catheter, and
an inflation lumen that run from the proximal end of the catheter to an inflation port distal to the delivery port, wherein the distal inflation port is proximal to the distal end of the catheter;
(ii) a balloon disposed about the catheter over the inflation port; and
(iii) an electrode disposed about the catheter proximal to the balloon, wherein the electrode is connected to the lead;
(b) positioning the electrode within the hollow tissue, wherein a region of the hollow tissue radially adjacent to the electrode comprises the target cell;
(c) flowing an inflation medium distally through the inflation lumen to inflate the balloon such that the balloon contacts the hollow tissue;
(d) introducing the agent through the delivery lumen and the delivery port such that the agent contacts the region of the hollow tissue radially adjacent to the electrode; and
(e) while the balloon is inflated, transmitting electrical energy through the electrode at conditions suitable for electrotransfer of the agent into the target cell.

56. The method of claim 53, wherein the agent is a therapeutic agent.

57. The method of claim 53, wherein the agent is a nucleic acid vector.

58. The method of claim 57, wherein the nucleic acid vector is expressed in the hollow tissue.

59. A method of expressing a nucleic acid vector in a target cell in a hollow tissue, the method comprising:
(a) providing a device comprising:
(i) a catheter comprising:
a proximal end and a distal end,
a delivery lumen that runs from the proximal end of the catheter to a delivery port proximal to the distal end of the catheter,
an elongate conductor that runs from the proximal end of the catheter to a lead proximal to the distal end of the catheter, and
an inflation lumens that run from the proximal end of the catheter to an inflation port distal to the delivery port, wherein the distal inflation port is proximal to the distal end of the catheter;
(ii) a balloon disposed about the catheter over the inflation port; and
(iii) an electrode disposed about the catheter proximal to the balloon, wherein the electrode is connected to the lead;
(b) positioning the electrode within the hollow tissue, wherein a region of the hollow tissue radially adjacent to the electrode comprises the target cell;
(c) flowing an inflation medium distally through the inflation lumen to inflate the balloon such that the balloon contacts the hollow tissue;
(d) introducing the nucleic acid vector through the delivery lumen and the delivery port such that the nucleic acid vector contacts the region of the hollow tissue radially adjacent to the electrode;
(e) while the balloon is inflated, transmitting electrical energy through the electrode at conditions suitable for electrotransfer of the nucleic acid vector into the target cell; and
(f) detecting expression of the nucleic acid vector by the target cell.

60. A method of treating a disease or disorder in an individual, the method comprising:
(a) providing a device comprising:
(i) a catheter comprising:
a proximal end and a distal end,
a delivery lumen that runs from the proximal end of the catheter to a delivery port proximal to the distal end of the catheter,
an elongate conductor that runs from the proximal end of the catheter to a lead proximal to the distal end of the catheter, and
an inflation lumens that run from the proximal end of the catheter to an inflation port distal to the delivery port, wherein the distal inflation port is proximal to the distal end of the catheter;
(ii) a balloon disposed about the catheter over the inflation port; and
(iii) an electrode disposed about the catheter proximal to the balloon, wherein the electrode is connected to the lead;
(b) positioning the electrode within a hollow tissue in the individual, wherein a region of the hollow tissue radially adjacent to the electrode comprises a target cell;
(c) flowing an inflation medium distally through the inflation lumen to inflate the balloon such that the balloon contacts the hollow tissue;
(d) introducing an effective amount of therapeutic agent through the delivery lumen and the delivery port such that the therapeutic agent contacts the region of the hollow tissue radially adjacent to the electrode; and
(e) while the balloon is inflated, transmitting electrical energy through the electrode at conditions suitable for electrotransfer of an effective amount of the therapeutic agent into the target cell, thereby treating the disease or disorder.

61. The method of claim 60, wherein the therapeutic agent is a nucleic acid vector.

62. The method of claim 57, wherein the nucleic acid vector comprises a therapeutic protein-encoding sequence.

63. The method of claim 62, wherein the therapeutic protein is a therapeutic replacement protein.

64. The method of claim 60, wherein the individual is monitored for progression of the disease or disorder after the treatment.

65. A method of exposing a target area in a hollow tissue to electrical energy, the method comprising:
(a) providing a device comprising:
(i) a catheter comprising:
a proximal end and a distal end;
a delivery lumen that runs from the proximal end of the catheter to a delivery port proximal to the distal end of the catheter;
an elongate conductor that runs from the proximal end of the catheter to a lead proximal to the distal end of the catheter; and
one or more inflation lumens that run from the proximal end of the catheter to a proximal inflation port proximal to the delivery port and a distal inflation port distal to the delivery port, wherein the distal inflation port is proximal to the distal end of the catheter;
(ii) a proximal balloon disposed about the catheter over the proximal inflation port;
(iii) a distal balloon disposed about the catheter over the distal inflation port; and
(iv) an electrode disposed about the catheter proximal to the balloon, wherein the electrode is connected to the lead;
(b) positioning the electrode within the hollow tissue, wherein the region of the hollow tissue radially adjacent to the electrode comprises all or a portion of the target area;
(c) flowing an inflation medium distally through the one or more inflation lumens to inflate the proximal balloon and the distal balloon such that each of the proximal balloon and distal balloon contacts the hollow tissue;
(d) introducing an agent through the delivery lumen and the delivery port such that the agent contacts the target area; and
(e) while the proximal and distal balloons are inflated, transmitting electrical energy through the electrode, thereby exposing the target area to the electrical energy.

66. The method of claim 65, wherein the electrical energy is transmitted at conditions suitable for electrotransfer of the agent into a target cell in the target area.

67. A method of delivering an agent to a target cell in a hollow tissue, the method comprising:
(a) providing a device comprising:
(i) a catheter comprising:
a proximal end and a distal end;
a delivery lumen that runs from the proximal end of the catheter to a delivery port proximal to the distal end of the catheter;
an elongate conductor that runs from the proximal end of the catheter to a lead proximal to the distal end of the catheter; and
one or more inflation lumens that run from the proximal end of the catheter to a proximal inflation port proximal to the delivery port and a distal inflation port distal to the delivery port, wherein the distal inflation port is proximal to the distal end of the catheter;
(ii) a proximal balloon disposed about the catheter over the proximal inflation port;
(iii) a distal balloon disposed about the catheter over the distal inflation port; and
(iv) an electrode disposed about the catheter proximal to the balloon, wherein the electrode is connected to the lead;
(b) positioning the electrode within the hollow tissue, wherein the region of the hollow tissue radially adjacent to the electrode comprises the target cell;
(c) flowing an inflation medium distally through the one or more inflation lumens to inflate the proximal balloon and the distal balloon such that each of the proximal balloon and distal balloon contacts the hollow tissue;
(d) introducing the agent through the delivery lumen and the delivery port such that the agent contacts the region of the hollow tissue radially adjacent to the electrode; and
(e) while the proximal and distal balloons are inflated, transmitting electrical energy through the electrode at conditions suitable for electrotransfer of the agent into the target cell.

68. The method of claim 65, wherein the agent is a therapeutic agent.

69. The method of claim 65, wherein the agent is a nucleic acid vector.

70. The method of claim 69, wherein the nucleic acid vector is expressed in the hollow tissue.

71. A method of expressing a nucleic acid vector in a target cell in a hollow tissue, the method comprising:
(a) providing a device comprising:
(i) a catheter comprising:
a proximal end and a distal end;
a delivery lumen that runs from the proximal end of the catheter to a delivery port proximal to the distal end of the catheter;
an elongate conductor that runs from the proximal end of the catheter to a lead proximal to the distal end of the catheter; and
one or more inflation lumens that run from the proximal end of the catheter to a proximal inflation port proximal to the delivery port and a distal inflation port distal to the delivery port, wherein the distal inflation port is proximal to the distal end of the catheter;
(ii) a proximal balloon disposed about the catheter over the proximal inflation port;
(iii) a distal balloon disposed about the catheter over the distal inflation port; and
(iv) an electrode disposed about the catheter proximal to the balloon, wherein the electrode is connected to the lead;
(b) positioning the electrode within the hollow tissue, wherein the region of the hollow tissue radially adjacent to the electrode comprises the target cell;
(c) flowing an inflation medium distally through the one or more inflation lumens to inflate the proximal balloon and the distal balloon such that each of the proximal balloon and distal balloon contacts the hollow tissue;
(d) introducing the nucleic acid vector through the delivery lumen and the delivery port such that the nucleic acid vector contacts the region of the hollow tissue radially adjacent to the electrode;
(e) while the proximal and distal balloons are inflated, transmitting electrical energy through the electrode at conditions suitable for electrotransfer of the nucleic acid vector into the target cell; and
(f) detecting expression of the nucleic acid vector by the target cell.

72. A method of treating a disease or disorder in an individual, the method comprising:
(a) providing a device comprising:
(i) a catheter, wherein the catheter comprises:
a proximal end and a distal end;
a delivery lumen that runs from the proximal end of the catheter to a delivery port proximal to the distal end of the catheter;
an elongate conductor that runs from the proximal end of the catheter to a lead proximal to the distal end of the catheter; and
one or more inflation lumens that run from the proximal end of the catheter to a proximal inflation port proximal to the delivery port and a distal inflation port distal to the delivery port, wherein the distal inflation port is proximal to the distal end of the catheter;
(ii) a proximal balloon disposed about the catheter over the proximal inflation port;
(iii) a distal balloon disposed about the catheter over the distal inflation port; and
(iv) an electrode disposed about the catheter proximal to the balloon, wherein the electrode is connected to the lead;
(b) positioning the electrode within a hollow tissue in the individual, wherein the region of the hollow tissue radially adjacent to the electrode comprises a target cell;
(c) flowing an inflation medium distally through the one or more inflation lumens to inflate the proximal balloon and the distal balloon such that each of the proximal balloon and distal balloon contacts the hollow tissue;
(d) introducing an effective amount of a therapeutic agent through the delivery lumen and the delivery port such that the therapeutic agent contacts the region of the hollow tissue radially adjacent to the electrode; and
(e) while the proximal and distal balloons are inflated, transmitting electrical energy through the electrode at conditions suitable for electrotransfer of an effective amount of the therapeutic agent into the target cell, thereby treating the disease or disorder.

73. The method of claim 72, wherein the therapeutic agent is a nucleic acid vector.

74. The method of claim 69, wherein the nucleic acid vector comprises a therapeutic protein-encoding sequence.

75. The method of claim 74, wherein the therapeutic protein is a therapeutic replacement protein.

76. The method of claim 72, wherein the individual is monitored for progression of the disease or disorder after the treatment.

77. The method of claim 72, wherein the one or more inflation lumens comprises a proximal inflation lumen that runs to the proximal inflation port and a distal inflation lumen that runs to the distal inflation port.

78. The method of claim 65, wherein step (c) comprises inflating the proximal balloon and the distal balloon simultaneously.

79. The method of claim 65, wherein step (c) comprises inflating the proximal balloon independently from the distal balloon.

80. The method of claim 79, wherein step (c) comprises inflating the proximal balloon after inflating the distal balloon.

81. The method of claim 65, wherein the catheter comprises a single inflation lumen that runs to both the proximal inflation port and the distal inflation port.

82. The method of claim 65, wherein the hollow tissue is a tubular tissue.

83. The method of claim 82, wherein the catheter further comprises a bypass lumen that runs from the proximal end of the catheter through the distal end of the catheter, wherein the method is performed without occluding flow through the tubular tissue.

84. The method of claim 82, wherein the hollow tissue is an airway.

85. The method of claim 84, wherein the airway is a trachea, a bronchus, or a bronchiole.

86. The method of claim 55, wherein the target cell is an airway epithelial cell.

87. The method of claim 53, wherein the agent is formulated as a liquid.

88. The method of claim 87, wherein the liquid is a viscous liquid.
